(19) United States
(12) Patent Application Publication
(10) Pub. No.: US 2022/0306714 A1
(43) Pub. Date: Sep. 29, 2022

(54) IL-2 FUSION PROTEINS THAT PREFERENTIALLY BIND IL-2RALPHA

(71) Applicant: ASKGENE PHARMA, INC., Camarillo, CA (US)

(72) Inventors: Chen YAO, Moorpark, CA (US); Chunxiao YU, Santa Barbara, CA (US); Yuefeng LU, Moorpark, CA (US); Liqin LIU, Woodland Hills, CA (US); Kurt SHANEBECK, Camarillo, CA (US); Shiwen ZHANG, Camarillo, CA (US)

(73) Assignee: ASKGENE PHARMA, INC., Camarillo, CA (US)

(21) Appl. No.: 17/634,890

(22) PCT Filed: Aug. 12, 2020
(86) PCT No.: PCT/US2020/046000

§ 371 Date: Feb. 11, 2022

Related U.S. Application Data
(60) Provisional application No. 62/885,471, filed on Aug. 12, 2019.
(60) Provisional application No. 63/015,644, filed on Apr. 26, 2020.
(60) Provisional application No. 63/019,319, filed on May 2, 2020.
(60) Provisional application No. 63/044,294, filed on Jun. 25, 2020.

(51) Int. Cl. C07K 14/55; C07K 14/715

(52) U.S. Cl. CPC C07K 14/55; C07K 14/7155; C07K 2319/32; C07K 2319/30; C07K 2319/50

(57) ABSTRACT

The present disclosure provides novel isolated IL-2 fusion molecules that preferentially activate regulatory T cells (Treg) in vitro and in vivo. Further included are methods of making and using said novel fusion molecules to treat inflammatory and autoimmune diseases.

### CROSS REFERENCE TO RELATED APPLICATIONS

The present application claims priority from U.S. Provisional Application No. 62/885,471, filed on Aug. 12, 2019; U.S. Provisional Application No. 63/015,644, filed on Apr. 26, 2020; U.S. Provisional Application No. 63/019,319, filed on May 2, 2020; and U.S. Provisional Application No. 63/044,294, filed on Jun. 25, 2020. The contents of the priority applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Aug. 12, 2020, is named 025471_WO005_SL.txt and is 163,708 bytes in size.

### BACKGROUND OF THE INVENTION

Interleukin-2 (IL-2) plays a central role in lymphocyte generation, survival and homeostasis. It has 133 amino acids and consists of four antiparallel, amphipathic alpha-helices that form a quaternary structure essential for its function (Smith, Science (1988) 240:1169-76; Bazan, Science (1992) 257:410-13). IL-2 exerts its activities by binding to IL-2 receptors (IL-2R), which consist of up to three individual subunits. Association of the α (CD25 or Tac antigen), β (CD122), and γ (γ_{c}, common γ chain, or CD132) subunits results in a trimeric, high-affinity receptor for IL-2 (K_{D}˜0.01 nM). Dimeric IL-2 receptor consisting of the β and γ subunits is termed intermediate-affinity IL-2R (K_{D}˜1 nM). The a subunit alone forms the monomeric low affinity IL-2 receptor (K_{D}˜10 nM). See, e.g., Kim et al., Cytokine Growth Factor Rev. (2006) 17:349-66). Although the dimeric intermediate-affinity IL-2 receptor binds IL-2 with approximately 100-fold lower affinity than the trimeric high-affinity receptor, both the dimeric and trimeric IL-2 receptors can transmit signal upon IL-2 binding (Minami et al., Annu Rev Immunol. (1993) 11:245-68). Thus, it appears that the a subunit, while helping to confer high-affinity binding of the receptor to IL-2, is not essential for IL-2 signaling. However, the β and γ subunits are essential for IL-2 signaling (Krieg et al., Proc Natl Acad Sci. (2010) 107:11906-11). The trimeric IL-2 receptor is expressed by CD4⁺FOXP3⁺ regulatory T (Treg) cells. Treg cells consistently express the highest level of IL-2Rα in vivo (Fontenot et al., Nature Immunol. (2005) 6:1142-51). The trimeric IL-2 receptor is also transiently induced on conventional activated T cells, whereas in the resting state these cells express only the dimeric IL-2 receptor.

Based on published crystal structures of IL-2/IL-2R complexes (Wang et al., Science (2005) 310:1159-63), researchers have made mutations in IL-2 to modulate its interaction with CD25, CD122, and/or CD132. In one example, it was reported that mutations at D20, N88 or Q126 of human IL-2 showed altered potency in activating T cells vs. NK cells (U.S. Pat. No. 6,955,807). In another example, it was shown that IL-2 with mutations at positions 69 and 74 bound to CD25 tightly, while mutations at position 88 or 91 interrupted its interaction with CD122, and mutations at position 126 interrupted its interaction with CD132 (PCT Publication WO 2009/061853).

Treg cells are essential for suppressing autoimmunity and regulating inflammation. FOXP3⁻CD25⁺ T effector cells (Teff) may be either CD4⁺ or CD8⁺ cells, both of which contribute to inflammation, autoimmunity, organ graft rejection, or graft-versus-host disease (GVHD). IL-2-stimulated STATS signaling is crucial for normal Treg cell growth and survival and for high FOXP3 expression.

Despite the role of IL-2 in Treg activities, there has been no clinically proven safe and efficacious IL-2-based therapy for regulating Treg activities. Thus, there remains a need to develop Il-2-based therapies that preferentially expand or stimulate Treg cells for treating inflammatory and autoimmune diseases.

### SUMMARY OF THE INVENTION

The present disclosure provides an isolated IL-2 fusion molecule, comprising a carrier moiety, a cytokine moiety, and one or more masking moieties, wherein the cytokine moiety is fused to the carrier moiety or to a masking moiety, the one or more masking moieties are fused to the carrier moiety or to the cytokine moiety, the cytokine moiety comprises an IL-2 polypeptide comprising (i) a C125A or C125S substitution, or (ii) an IL-2 amino acid sequence comprising one or more substitutions selected from T3A, C125S, V69A, and Q74P (numbering according to SEQ ID NO: 1), the one or more masking moieties bind to the cytokine moiety and inhibit binding of the cytokine moiety to IL-2Rβ and/or IL-2Rγ, but not to IL-2α, on immune cells (e.g., T cells and NK cells). In some embodiments, the IL-2 polypeptide binds to IL-2Rα with an affinity similar to or higher than wildtype IL-2.

The present disclosure also provides a method of treating an inflammatory condition or an autoimmune disease, comprising administering to a subject in need thereof a therapeutically amount of an isolated IL-2 fusion molecule comprising a carrier moiety, a cytokine moiety and one or more masking moieties, wherein the cytokine moiety is fused to the carrier moiety or to a masking moiety, the one or more masking moieties are fused to the carrier moiety or to the cytokine moiety, the cytokine moiety comprises an IL-2 polypeptide, and the one or more asking moieties bind to the cytokine moiety and inhibit binding of the cytokine moiety to IL-2Rβ and/or IL-2Rγ, but not to IL-2α, on immune cells (e.g., T cells and NK cells). In some embodiments, the inflammatory condition or autoimmune disease is selected from the group consisting of asthma, Type I diabetes, rheumatoid arthritis, allergy, systemic lupus erythematosus, multiple sclerosis, organ graft rejection, and graft-versus-host disease.

In some embodiments, the IL-2 polypeptide binds to IL-2Rα with an affinity similar to or higher than that of wildtype IL-2.

In some embodiments, IL-2Rβ ECD or its functional analog has an amino acid sequence at least 95% (e.g., at least 97%, at least 98%, or at least 99%) identical to SEQ ID NO: 3. In some embodiments, the IL-2Rγ ECD or its functional analog has an amino acid sequence at least 95% (e.g., at least 97%, at least 98%, or at least 99%) identical to SEQ ID NO: 6. In some embodiments, the IL-2 polypeptide comprises an amino acid sequence that is at least 95% identical to SEQ ID NO:1, optionally wherein the amino acid sequence is SEQ ID NO: 2.

In some embodiments, the IL-2 fusion molecule comprises a masking moiety comprising an extracellular domain (ECD) of IL-2Rβ or IL-2Rγ, or a functional analog thereof, wherein the masking moiety is fused to the carrier moiety with or without a peptide linker. In other embodiments, the IL-2 fusion molecule comprises a first masking moiety comprising an extracellular domain (ECD) of IL-2Rβ or IL-2Rγ, or a functional analog thereof, wherein the first masking moiety is fused to the carrier moiety with or without a peptide linker, and a second masking moiety comprising an ECD of IL-2Rγ or IL-2Rβ, or a functional analog thereof, wherein the second masking moiety is fused to the cytokine moiety or to the first masking moiety with or without a peptide linker. In some embodiments, the IL-2 fusion molecule of the present disclosure comprises at least two masking moieties, one of which is an ECD of IL-2Rα or a functional analog thereof, wherein the IL-2Rα ECD masking moiety is fused to the cytokine moiety, the carrier moiety, or another masking moiety through a cleavable peptide linker. In particular embodiments, the IL-2Rα ECD moiety comprises an amino acid sequence at least 95% identical to SEQ ID NO: 7.

In some embodiments, the cytokine moiety is fused to the carrier moiety or a masking moiety through a non-cleavable peptide linker, and the masking moiety is fused to the carrier moiety or the cytokine moiety through a non-cleavable peptide linker. In particular embodiments, the masking moiety is fused to the carrier moiety or the cytokine moiety through a peptide linker comprising at least 16 amino acids, at least 18 amino acids, at least 20 amino acids, at least 22 amino acids, at least 25 amino acids, at least 30, or up to 44 amino acids.

In some embodiments, the carrier moiety is selected from a PEG molecule, an albumin, an albumin fragment, an antibody Fc domain, an antibody, or an antigen-binding fragment thereof. In some embodiments, the carrier moiety is an antibody Fc domain, and the fusion molecule is a heterodimer comprising a first polypeptide chain comprising, from N-terminus to C-terminus, a molecular formula selected from F1-L1-E1, F1-L1-E1-L2-E2, and F1-L1-E2-L2-E1, and a second polypeptide chain comprising, from N-terminus to C-terminus, a molecular formula F2-L3-C, wherein F1 and F2 are the subunits of the Fc domain, L1, L2 and L3 are peptide linkers, E1 is an IL-2Rβ ECD or a functional analog thereof, and E2 is an IL-2Rγ ECD or a functional analog thereof, and C is the cytokine moiety. In other embodiments, the carrier moiety is an antibody Fc domain, and wherein the fusion molecule is a heterodimer comprising a first polypeptide chain comprising, from N-terminus to C-terminus, a molecular formula selected from E1-L1-F1, E1-L1-E2-L2-F1, and E2-L1-E1-L2-F1, and a second polypeptide chain comprising, from N-terminus to C-terminus, a molecular formula C-L3-F2, wherein F1 and F2 are the subunits of the Fc domain, L1, L2 and L3 are peptide linkers, E1 is an IL-2Rβ ECD or a functional analog thereof, and E2 is an IL-2Rγ ECD or a functional analog thereof, and C is the cytokine moiety. In other embodiments, the carrier moiety is an antibody Fc domain, and wherein the fusion molecule is a heterodimer comprising a first polypeptide chain and a second polypeptide chain comprising, from N-terminus to C-terminus, molecular formulae selected from the following pairs:

F1-L1-E1 and F2-L2-C-L3-E2,

F1-L1-E1 and F2-L2-E2-L3-C,

F1-L1-E2 and F2-L2-C-L3-E1,

F1-L1-E2 and F2-L2-E1-L3-C,

E1-L1-F1 and E2-L2-C-L3-F2,

E1-L1-F1 and C-L2-E2-L3-F2,

E2-L1-F1 and E2-L2-C-L3-F2, and

E2-L1-F1 and C-L2-E1-L3-F2, wherein

F1 and F2 are the subunits of the Fc domain, L1, L2 and L3 are peptide linkers, E1 is an IL-2Rβ ECD or a functional analog thereof, and E2 is an IL-2Rγ ECD or a functional analog thereof, and C is the cytokine moiety. In some embodiments, the peptide linkers L1, L2, and L3 are not cleavable. In particular embodiments, L1, L2, and L3 independently have an amino acid sequence selected from SEQ ID NOs: 40-46, 55-57 and 59. In other particular embodiments, at least one of L1, L2, and L3 has an amino acid sequence comprising 20-44 amino acids.

In particular embodiments, the IL-2 fusion molecule of the present disclosure comprises a first polypeptide chain comprising an amino acid sequence at least 99% identical to SEQ ID NO: 50, 51, or 52, and a second polypeptide chain comprising an amino acid sequence at least 99% identical to SEQ ID NO: 53 or 54. In a particular embodiments, the IL-2 fusion molecule of the present disclosure comprises a first polypeptide chain comprising an amino acid sequence at least 99% identical to SEQ ID NO: 50, and a second polypeptide chain comprising an amino acid sequence at least 99% identical to SEQ ID NO: 53.

In some embodiments, the IL-2 fusion molecule of the present disclosure has one or more of the following properties:
(a) binds to high affinity IL-2 receptor with alpha, beta, and gamma subunits (IL-2Rαβγ) with an affinity that is at least 100 times higher than that of intermediate IL-2 receptor with beta and gamma subunits (IL-2Rβγ),
(b) binds to IL-2Rβγ with a KD of more than about 5 nM or more than 10 nM as measured in a surface plasmon resonance assay at 37° C.,
(c) has an EC50 value of less than about 1 nM and greater than 0.01 nM, 0.25 nM, or 0.05 nM in a CTLL-2 cell proliferation assay,
(d) has an EC50 value of greater than about 0.05 nM, 0.1 nM, 0.25 nM, or 0.5 nM in a NK92 cell proliferation assay,
(e) has an Emax value at least 5 times or at least 10 times lower in a NK92 cell proliferation assay in the presence of a neutralizing CD25 antibody than in the absence of the neutralizing CD25 antibody,
(f) preferentially stimulates FOXP3+ T regulatory cells relative to T effector cells or NK cells,
(g) promotes FOXP3+ regulatory T cell growth or survival, and
(h) induces STATS phosphorylation in FOXP3+ T cells but has a reduced ability to induce phosphorylation of STATS in FOXP3− T cells.

In other aspects, the present disclosure provides also a pharmaceutical composition comprising the IL-2 fusion molecule of the present disclosure and a pharmaceutically acceptable excipient; a polynucleotide or polynucleotides encoding the IL-2 fusion molecule, an expression vector or vectors comprising the polynucleotide or polynucleotides; and a host cell comprising the vector(s), wherein the host cell may be a prokaryotic cell or a eukaryotic cell such as a mammalian cell. In some embodiments, the mammalian host cell has the gene or genes encoding uPA, MMP-2 and/or MMP-9 knocked out (e.g., containing null mutations of one or more of these genes). Accordingly, the present disclosure also provides a method of making the IL-2 fusion molecule, comprising culturing the host cell under conditions that allow expression of the IL-2 fusion molecule, wherein the host cell is a mammalian cell, and isolating the IL-2 fusion molecule.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTIONS OF THE DRAWINGS

FIGS. 1A and 1B are schematic illustrations of IL-2 fusion molecules with an IL-2Rβ extracellular domain (ECD) and an IL-2 polypeptide fused to the C-termini of an Fc domain. The IL-2 polypeptide is fused to the C-terminus of one Fc polypeptide via a noncleavable linker. The IL-2Rβ ECD is fused to the C-terminus of the other Fc polypeptide via a noncleavable linker (FIG. 1A) or a cleavable linker (FIG. 1B). “Knobs-into-holes” indicate knobs-into-holes mutations in the Fc polypeptides.

FIGS. 2A and 2B are schematic illustrations of IL-2 fusion molecules with an IL-2Rβ ECD and an IL-2 polypeptide fused to the C-termini of a Fc domain and an IL-2Rγ ECD fused to the C-terminus of the IL-2Rβ ECD. The IL-2 polypeptide is fused to the C-terminus of one Fc polypeptide via a noncleavable linker. The IL-2Rβ ECD is fused to the C-terminus of the other Fc polypeptide via a noncleavable linker. The IL-2Rγ ECD is fused to the C-terminus of the IL-2Rβ ECD via a noncleavable linker (FIG. 2A) or a cleavable linker (FIG. 2B).

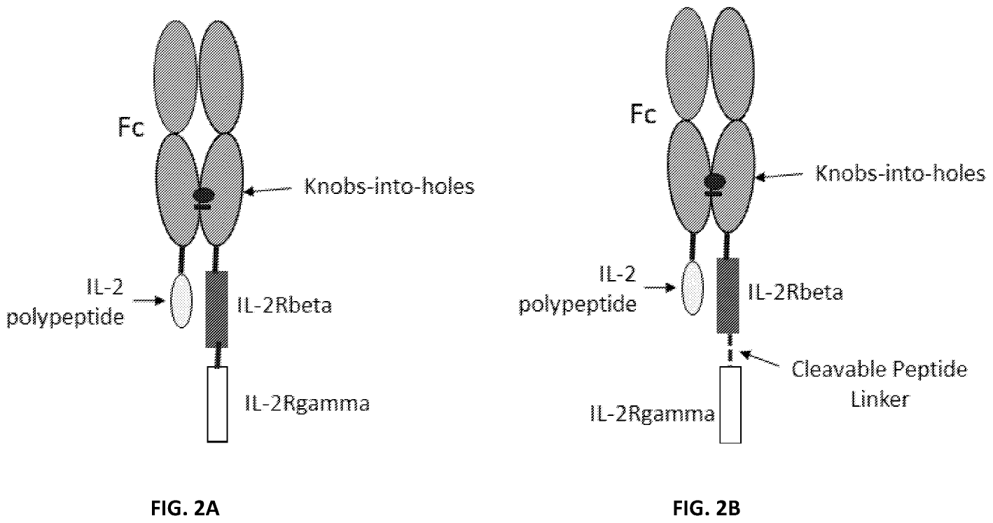

FIGS. 3A and 3B are schematic illustrations of IL-2 fusion molecules with an IL-2Rγ ECD and an IL-2 polypeptide fused to the C-termini of an Fc domain, and an IL-2Rβ ECD fused to the C-terminus of the IL-2Rγ ECD. The IL-2 polypeptide is fused to the C-terminus of one Fc polypeptide via a noncleavable linker. The IL-2Rγ ECD is fused to the C-terminus of the other Fc polypeptide via a noncleavable linker. The IL-2Rβ ECD is fused to the C-terminus of the IL-2Rγ ECD via a noncleavable linker (FIG. 3A) or a cleavable linker (FIG. 3B).

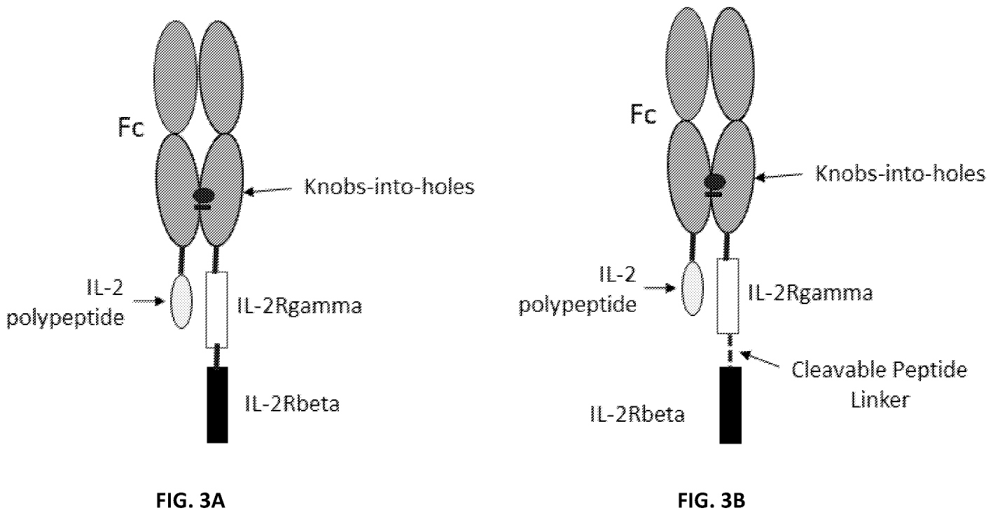

FIGS. 4A and 4B are schematic illustrations of IL-2 fusion molecules with an IL-2Rβ ECD and an IL-2 polypeptide fused to the C-termini of an Fc domain, and an IL-2Rγ ECD fused to the C-terminus of the IL-2 polypeptide. The IL-2 polypeptide is fused to the C-terminus of one Fc polypeptide via a noncleavable linker. The IL-2Rγ ECD is fused to the C-terminus of the IL-2 polypeptide via a cleavable linker. The IL-2Rβ ECD is fused to the C-terminus of the other Fc polypeptide via a noncleavable linker (FIG. 4A) or a cleavable linker (FIG. 4B).

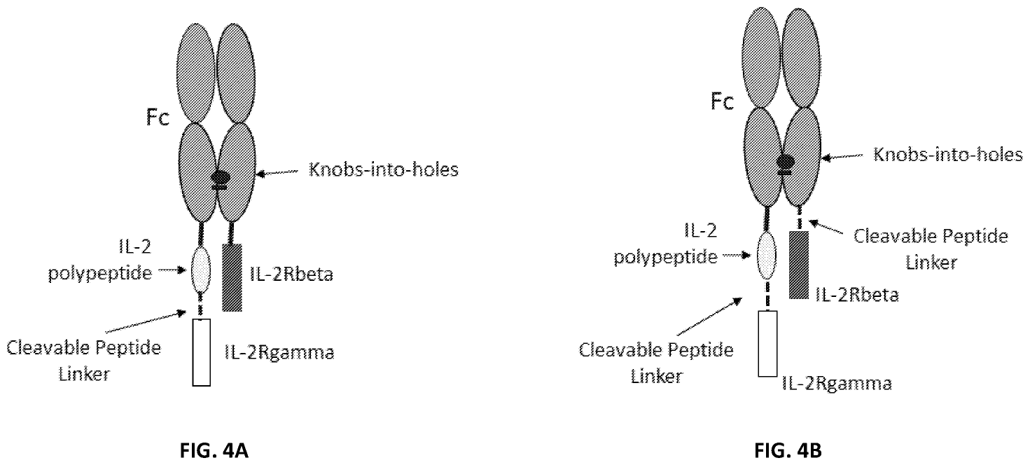

FIGS. 5A and 5B are schematic illustrations of IL-2 fusion molecules with an IL-2Rγ ECD and an IL-2 polypeptide fused to the C-termini of an Fc domain, and an IL-2Rβ ECD fused to the C-terminus of the IL-2 polypeptide. The IL-2 polypeptide is fused to the C-terminus of one Fc polypeptide via a noncleavable linker. The IL-2Rβ is fused to the C-terminus of the IL-2 polypeptide via a cleavable linker. The IL-2Rγ ECD is fused to the C-terminus of the other Fc polypeptide via a noncleavable linker (FIG. 5A) or a cleavable linker (FIG. 5B).

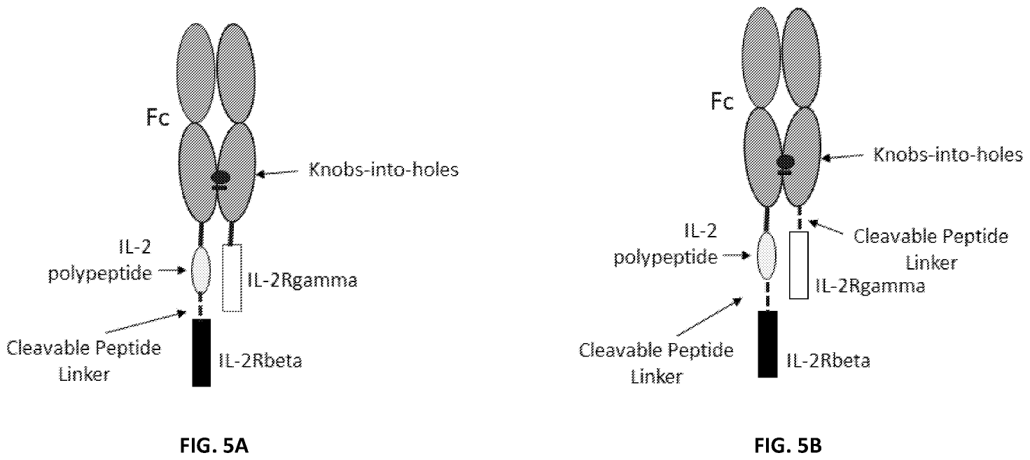

FIGS. 6A and 6B are schematic illustrations of IL-2 fusion molecules with an IL-2Rβ ECD and an IL-2Rγ ECD fused to the C-termini of an Fc domain, and an IL-2 polypeptide fused to the C-terminus of the IL-2Rβ ECD or the IL-2Rγ ECD. In FIG. 6A, the IL-2Rγ ECD is fused to the C-terminus of one Fc polypeptide via a cleavable linker, the IL-2Rβ ECD is fused to the C-terminus of the other Fc polypeptide via a noncleavable linker, and the IL-2 polypeptide is fused to the C-terminus of the IL-2Rβ ECD via a noncleavable linker. In FIG. 6B, the IL-2Rβ ECD is fused to the C-terminus of one Fc polypeptide via a cleavable linker, the IL-2Rγ ECD is fused to the C-terminus of the other Fc polypeptide via a noncleavable linker, and the IL-2 polypeptide is fused to the C-terminus of the IL-2Rγ ECD via a noncleavable linker.

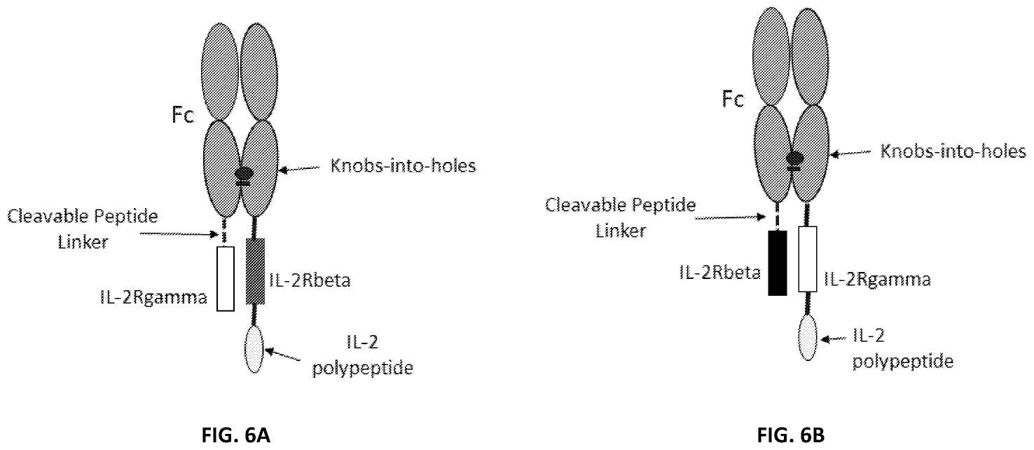

FIGS. 7A and 7B are schematic illustrations of IL-2 fusion molecules with an IL-2Rβ ECD and an IL-2 polypeptide fused to the N-termini of the Fc domain. The IL-2 polypeptide is fused to the N-terminus of one Fc polypeptide. The IL-2Rβ ECD is fused to the N-terminus of the other Fc polypeptide via a noncleavable linker (FIG. 7A) or a cleavable linker (FIG. 7B).

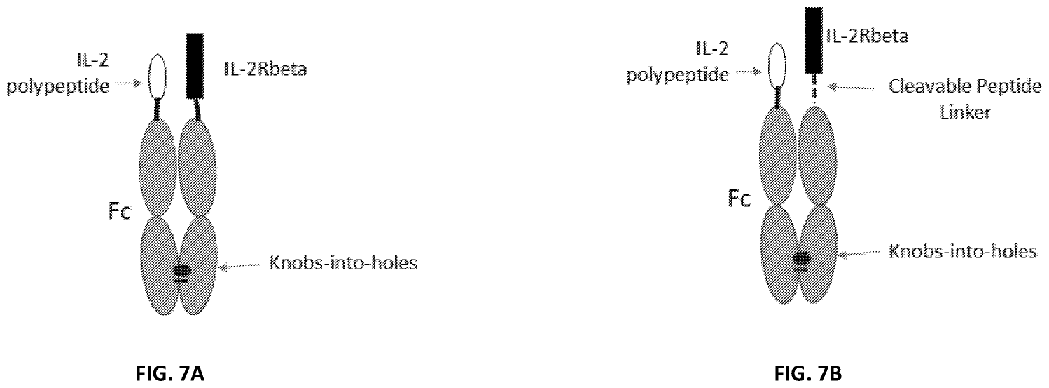

FIG. 8 shows SDS-PAGE analysis of the IL-2 fusion molecule JR3.116.5 with a schematic structure as illustrated in FIG. 1B, which comprises two polypeptide chains with amino acid sequences as shown in SEQ ID NOs: 12 and 23, respectively.

FIG. 9 shows the results of CTLL2-based biological activity assay of the IL-2 fusion molecule JR3.116.5 prior to and after activation by a protease treatment.

FIGS. 10A and 10B are schematic illustrations of IL-2 fusion molecules 982 C1, 982 C2, 982 D1 and 982 D2. 982 C1 and 982 C2 have two masking moieties, IL-2Rβ ECD and IL-2Rγ ECD, and an IL-2 mutein is fused to the C-termini of an IgG₄ Fc domain. FIG. 10A shows an IL-2Rγ ECD fused to the C-terminus of one IgG₄ Fc polypeptide via a (G₄S)₂AA(G₄S)₂ (SEQ ID NO: 59) noncleavable linker. The IL-2Rβ ECD is fused to the C-terminus of the IL-2Rγ ECD via a 43 amino acid long noncleavable linker as shown in SEQ ID NO: 46). The IL-2 mutein is fused to the C-terminus of the other IgG₄ polypeptide via a noncleavable linker. The IL-2 mutein has a C125A substitution (982 C1) or substitutions T3A/C125SN69A/Q74P (982 C2). FIG. 10B shows an IL-2Rβ ECD fused to the C-terminus of one IgG₄ Fc polypeptide via a (G₄S)₂AA(G₄S)₂ (SEQ ID NO: 59) noncleavable linker. The IL-2 mutein is fused to the C-terminus of the other IgG₄ polypeptide via a noncleavable linker. The IL-2 mutein has a C125A substitution (982 D1) or the substitutions T3A/C125S/V69A/Q74P (982 D2).

FIG. 11 shows the NK92 cell proliferation assay of the 982 D1, 982 D2, IL-2, and a reference molecule with in the presence or absence of a neutralizing antibody against CD25. The reference molecule (982 Ref) is an Fc-IL-2 fusion molecule with IL-2 having mutations V91K and C125A. 982-Ref is a homodimer Fc-fusion-IL-2 mutein molecule with each chain comprising an amino acid sequence of SEQ ID NO: 58.

FIG. 12 shows the binding of IL-2 fusion molecules 982 D1 and 982 D2 to rat CD4⁺ T cells. N.C. represents buffer control.

FIGS. 13A and 13B show the binding of IL-2 fusion molecules 982 D1, 982 C1, and 982 D2, and 982 Ref to CD4⁺CD25⁺ T cells and CD4⁺CD25⁻ T cells. N.C. represents buffer control.

FIG. 14 shows the concentration-dependent proliferation of CD4⁺CD25⁺ T cells and CD4⁺CD25⁻ T cells induced by the 982 D1, 982 C1, 982 D2, and 982 Ref IL-2 fusions molecules. IL-2 alone was also tested.

FIG. 15 shows the serum plasma concentration of 982 C1, 982 D1, and 982 Ref IL-2 fusion molecules over time in a rat PK study. The rats were injected with the molecules subcutaneously.

FIG. 16 shows the serum plasma concentration of 982 C1, 982 D1, and 982 Ref IL-2 fusion molecules over time from a second rat PK study.

FIGS. 17A and 17B show changes over time in the percentage of CD4⁺FOXP3⁺ and CD4⁺FOXP3⁻ cells among the CD4⁺ T cells in rats induced by 982 C1, 982 D1, and 982 Ref IL-2 fusion molecules.

FIGS. 18A and 18B show changes over time in proliferation status (as indicated by proliferation marker Ki67) of CD4⁺CD25⁺ and CD4⁺CD25⁻ cells induced by 982 C1, 982 D1, and 982 Ref in rats from the first PK study.

FIGS. 19A and 19B show changes over time in the percentage of in CD4⁺FOXP3⁺ and CD4⁺FOXP3⁻ cells induced by 982 IL-2 fusion molecules among the CD4⁺ T cells in rats.

FIGS. 20A and 20B show changes over time in proliferation status of CD4⁺CD25⁺ and CD4⁺CD25⁻ cells induced by 982 IL-2 fusion molecules in rats.

FIG. 21 shows the body weights of the various treatment groups after a single subcutaneous administration of 982 D1, 982 D2, and 982 Ref.

### DETAILED DESCRIPTION OF THE INVENTION

As used herein and in the appended claims, the singular forms “a,” “or,” and “the” include plural referents unless the context clearly dictates otherwise. Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “antigen-binding moiety” refers to a polypeptide or a set of interacting polypeptides that specifically bind to an antigen, and includes, but is not limited to, an antibody (e.g., a monoclonal antibody, polyclonal antibody, a multi-specific antibody, a dual specific or bispecific antibody, an anti-idiotypic antibody, or a bifunctional hybrid antibody) or an antigen-binding fragment thereof (e.g., a Fab, a Fab′, a F(ab′)₂, a Fv, a disulfide linked Fv, a scFv, a single domain antibody (dAb), or a diabody), a single chain antibody, and an Fc-containing polypeptide such as an immunoadhesin. In some embodiments, the antibody may be of any heavy chain isotype (e.g., IgG, IgA, IgM, IgE, or IgD) or subtype (e.g., IgG₁, IgG₂, IgG₃, or IgG₄). In some embodiments, the antibody may be of any light chain isotype (e.g., kappa or lambda). The antibody may be human, non-human (e.g., from mouse, rat, rabbit, goat, or another non-human animal), chimeric (e.g., with a non-human variable region and a human constant region), or humanized (e.g., with non-human CDRs and human framework and constant regions). In some embodiments, the antibody is a derivatized antibody.

The term “cytokine agonist polypeptide” refers to a wildtype cytokine, or an analog thereof. An analog of a wildtype cytokine has the same biological specificity (e.g., binding to the same receptor(s) and activating the same target cells) as the wildtype cytokine, although the activity level of the analog may be different from that of the wildtype cytokine. The analog may be, for example, a mutein (i.e., mutated polypeptide) of the wildtype cytokine, and may comprise at least one, at least two, at least three, at least four, at least five, at least six, at least seven, at least eight, at least nine, or at least ten mutations relative to the wildtype cytokine.

The term “cytokine antagonist” or “cytokine mask” refers to a moiety (e.g., a polypeptide) that binds to a cytokine, thereby inhibiting the cytokine from binding to its receptor on the surface of a target cell and/or exerting its biological functions while being bound by the antagonist or mask. Examples of a cytokine antagonist or mask include, without limitations, a polypeptide derived from an extracellular domain of the cytokine's natural receptor that makes contact with the cytokine.

The term “effective amount” or “therapeutically effective amount” refers to an amount of a compound or composition sufficient to treat a specified disorder, condition, or disease, such as ameliorate, palliate, lessen, and/or delay one or more of its symptoms.

The term “functional analog” refers to a molecule that has the same biological specificity (e.g., binding to the same ligand) and/or activity (e.g., activating or inhibiting a target cell) as a reference molecule.

The term “fused” or “fusion” in reference to two polypeptide sequences refers to the joining of the two polypeptide sequences through a backbone peptide bond. Two polypeptides may be fused directly or through a peptide linker that is one or more amino acids long. A fusion polypeptide may be made by recombinant technology from a coding sequence containing the respective coding sequences for the two fusion partners, with or without a coding sequence for a peptide linker in between. In some embodiments, fusion encompasses chemical conjugation.

The term “pharmaceutically acceptable excipient” when used to refer to an ingredient in a composition means that the excipient is suitable for administration to a treatment subject, including a human subject, without undue deleterious side effects to the subject and without affecting the biological activity of the active pharmaceutical ingredient (API).

The term “subject” refers to a mammal and includes, but is not limited to, a human, a pet (e.g., a canine or a feline), a farm animal (e.g., cattle or horse), a rodent, or a primate.

As used herein, “treatment” or “treating” is an approach for obtaining beneficial or desired clinical results. Beneficial or desired clinical results include, but are not limited to, one or more of the following: alleviating one or more symptoms resulting from a disease, diminishing the extent of a disease, ameliorating a disease state, stabilizing a disease (e.g., preventing or delaying the worsening or progression of the disease), preventing or delaying the spread (e.g., metastasis) of a disease, preventing or delaying the recurrence of a disease, providing partial or total remission of a disease, decreasing the dose of one or more other medications required to treat a disease, increasing the patient's quality of life, and/or prolonging survival. The methods of the present disclosure contemplate any one or more of these aspects of treatment.

It is to be understood that one, some or all of the properties of the various embodiments described herein may be combined to form other embodiments of the present invention. The section headings used herein are for organizational purposes only and are not to be construed as limiting the subject matter described thereunder.

### Isolated IL-2 Fusion Molecules

The present disclosure provides IL-2 fusion molecules that are useful for the treatment of inflammatory and autoimmune diseases. The inventors were surprised that the desired in vivo activities were achieved without the need for cleavage or removal of the masking moiety. The masked IL-2 fusion molecules with non-cleavable peptide linkers possess a number of significant advantages compared to cleavable masked IL-2 fusion molecules. For example, the cleavable masked IL-2 molecules need protease cleavage of a linker and removal of the masking moiety in order to be activated. Due to uneven distribution of the protease(s) at the disease site, its level of cytokine activation would variate, which could add variability to therapeutic efficacy. In addition, non-specific activations in circulation and/or during the production may also take place, adding safety concerns and production complexity to the cleavable masked molecules.

In some embodiments, the IL-2 fusion molecules of the present disclosure have reduced affinity (e.g., with a K_{D} high than 1 nM, higher than 5 nM, higher than 10 nM, higher than 100 nM, or higher than 1 μM) for intermediate affinity IL-2Rβγ, while retaining the wildtype affinity (e.g., a K_{D} of about 10 nM) for IL-2Rα (CD25), or having an affinity similar to (e.g., with a K_{D} of about 1-20 nM), even higher than (e.g., with a K_{D} lower than 10 nM, lower than 5 nM, or lower than 1 nM), the wildtype affinity for IL-2Rα. An isolated IL-2 fusion molecule may comprise an IL-2 polypeptide (cytokine moiety), a carrier (carrier moiety), and an IL-2 antagonist (masking moiety or cytokine antagonist), wherein the IL-2 polypeptide is fused to the carrier directly or through a cleavable or non-cleavable peptide linker, and the IL-2 antagonist is linked to the IL-2 polypeptide or to the carrier through a non-cleavable or cleavable peptide linker. In some embodiments, the cytokine moiety may be fused to a masking moiety, which may be fused to the carrier moiety directly or through a cleavable or noncleavable linker.

In preferred embodiments, the IL-2 polypeptide is fused to the carrier through a non-cleavable peptide linker, and the IL-2 antagonist is linked to the carrier or the IL-2 polypeptide through a non-cleavable peptide linker. For example, the IL-2 antagonist may be fused to the carrier through the non-cleavable peptide linker of SEQ ID NO: 59. In some embodiments, the IL-2 polypeptide is a wildtype IL-2 polypeptide or does not comprise a mutation that reduces the polypeptide's binding affinity for CD25.

The present IL-2 fusion molecules may comprise an IL-2 polypeptide (cytokine moiety) linked to a carrier moiety and masked (bound) by a cytokine antagonist (masking moiety). The cytokine antagonist is selected from the extracellular domain (ECD) of IL-2Rβ (CD122), a functional analog of IL-2Rβ ECD, IL-2Rγ ECD (CD132), a functional analog of IL-2Rγ ECD, and a combination of IL-2Rβ ECD and IL-2Rγ ECD. In some embodiments, the cytokine antagonist inhibits the binding of the cytokine moiety to IL-2Rγ and/or of IL-2Rβ on T cells in a patient in need thereof, while the cytokine moiety to bind to IL-2Rα (CD25) remains intact. Because IL-2Rα (CD25) is preferentially expressed on Treg cells, the present IL-2 fusion molecules can preferentially stimulate the proliferation of Treg cells, while having minimal effect on non-Treg cells.

In some embodiments, the carrier moiety is an Fc domain. In some embodiments, the present IL-2 fusion molecule is a heterodimer comprising a first polypeptide chain comprising, from N-terminus to C-terminus, a molecular formula selected from F1-L1-E1, F1-L1-E1-L2-E2, and F1-L1-E2-L2-E1, and a second polypeptide chain comprising, from N-terminus to C-terminus, a molecular formula F2-L3-C, wherein F1 and F2 are the subunits of a heterodimeric Fc domain, L1, L2 and L3 are peptide linkers, E1 is an IL-2Rβ ECD or its functional analog, E2 is an IL-2Rγ ECD or its functional analog, and C is a cytokine moiety comprising an IL-2 polypeptide (e.g., wildtype human IL-2 or a mutein thereof).

In some embodiments, the present IL-2 fusion molecule is a heterodimer comprising a first polypeptide chain comprising, from N-terminus to C-terminus, a molecular formula selected from E1-L1-F1, E1-L1-E2-L2-F1, and E2-L1-E1-L2-F1, and a second polypeptide chain comprising, from N-terminus to C-terminus, a molecular formula C-L3-F2, wherein F1 and F2 are the subunits of a heterodimeric Fc domain, L1, L2 and L3 are peptide linkers, E1 is an IL-2Rβ ECD or its functional analog, E2 is an IL-2Rγ ECD or its functional analog, and C is a cytokine moiety comprising an IL-2 polypeptide (e.g., wildtype human IL-2 or a mutein thereof).

In some embodiments, the present IL-2 fusion molecule is a heterodimer comprising a first polypeptide chain and a second polypeptide chain comprising, from N-terminus to C-terminus, molecular formulae selected from the following pairs:

a. F1-L1-E1 and F2-L2-C-L3-E2;

b. F1-L1-E1 and F2-L2-E2-L3-C;

c. F1-L1-E2 and F2-L2-C-L3-E1;

d. F1-L1-E2 and F2-L2-E1-L3-C;

e. E1-L1-F1 and E2-L2-C-L3-F2;

f. E1-L1-F1 and C-L2-E2-L3-F2;

g. E2-L1-F1 and E2-L2-C-L3-F2; and

h. E2-L1-F1 and C-L2-E1-L3-F2;

wherein F1 and F2 are the subunits of a heterodimeric Fc domain, L1, L2 and L3 are peptide linkers, E1 is IL-2Rβ ECD or its functional analog, E2 is IL-2Rγ ECD or its functional analog, and C is the cytokine moiety.

In some embodiments, the peptide linkers L1, L2, and L3 independently have an amino acid sequence selected from SEQ ID NOs: 40-49 and 55-57.

In some embodiments, at least one of the peptide linkers L1, L2, and L3 has an amino acid sequence that comprises at least 20-44 amino acids (e.g., at least 20, at least 25, at least 30, at least 35, at least 40, at least 45, or at least 55). In some embodiments, at least one of the peptide linkers has at least 16, 18, 20, 22, 24, 26, 27, 28, 29, 31, 32, 33, 34, 36, 37, 38, 39, 41, or 42 amino acids.

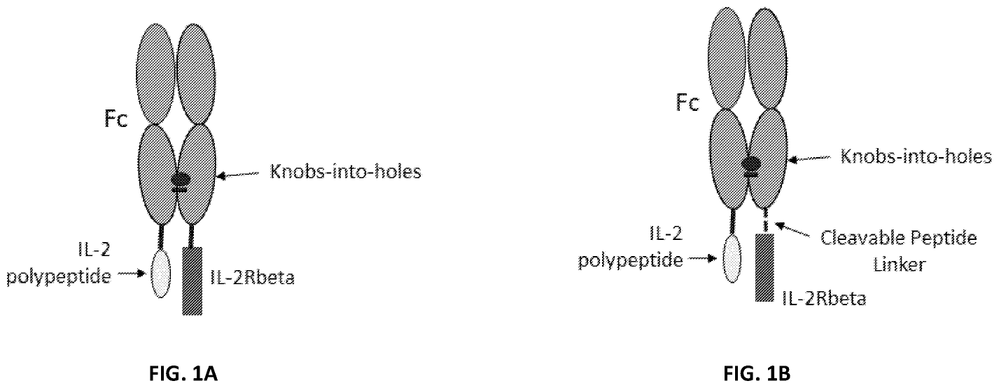

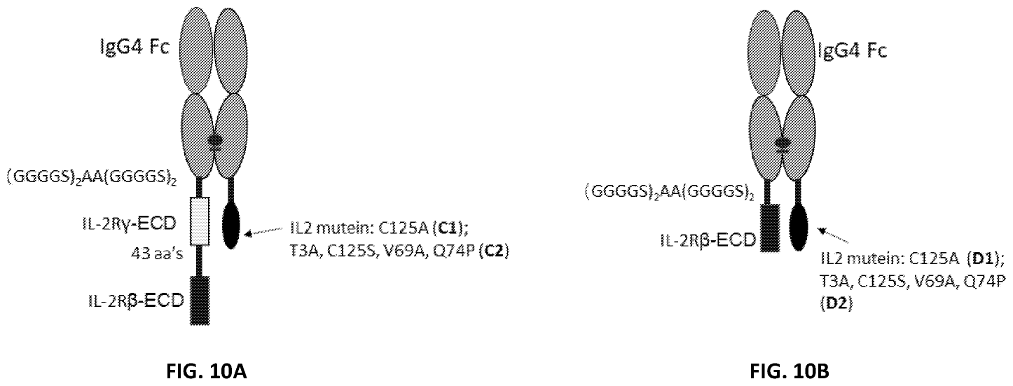

In some embodiments, the present IL-2 fusion molecule has a structure as illustrated in FIG. 1A, 1B, 2A, 2B, 3A, 3B, 4A, 4B, 5A, 5B, 6A, 6B, 7A, or 7B. In particular embodiments, the IL-2 fusion molecule has a structure illustrated in FIG. 10A or 10B.

In some embodiments, the isolated fusion molecule comprises a first polypeptide chain comprising an amino acid sequence at least 99% identical to one selected from SEQ ID NO: 50, 51 and 52, and a second polypeptide chain with an amino acid sequence at least 99% identical to one selected from SEQ ID NOs: 53 and 54.

The isolated IL-2 fusion molecules 982 C1, C2, D1, D2 and 982 Ref comprise two polypeptide chains with amino acid sequences as shown in Table 1. Both 982 C1 and 982 C2 molecules comprise two masking moieties, which are an IL-2Rγ ECD and an IL-2Rβ ECD. Both 982 D1 and 982 D2 each comprise one masking moiety, which is an IL-2Rβ ECD. The IL-2 moiety of both 982 C2 and 982 D2 comprises mutations T3A, V69A, P74Q, and C125S (numbering according to SEQ ID NO: 1).

TABLE 1
Sequences of 982 C1, C2, D1, D2 and 982 Ref

| Molecule Name | Polypeptide Chain 1 | Polypeptide Chain 2 |
|---|---|---|
| 982 C1 | SEQ ID NO: 50 | SEQ ID NO: 53 |
| 982 C2 | SEQ ID NO: 52 | SEQ ID NO: 54 |
| 982 D1 | SEQ ID NO: 50 | SEQ ID NO: 53 |
| 982 D2 | SEQ ID NO: 52 | SEQ ID NO: 54 |
| 982 Ref | SEQ ID NO: 58 | SEQ ID NO: 58 |

A. IL-2 Polypeptide or Mutein

In the present IL-2 fusion molecules, the IL-2 polypeptide may be a wildtype IL-2 polypeptide such as a wildtype human IL-2 polypeptide (SEQ ID NO: 1), or an IL-2 mutein such as an IL-2 mutein derived from a human IL-2. An IL-2 mutein is an IL-2 derivative that retains at least one or more aspects of the IL-2 biological activities. In some embodiments, IL-2 mutein comprises a sequence of amino acids at least 95% identical to SEQ ID NO: 1. In certain embodiments, the IL-2 mutein has the same length as SEQ ID NO: 1 but differs from it by no more than 7 (e.g., no more than 6, no more than 5, no more than 4, no more than 3, or no more than 2) amino acid residues. The IL-2 mutein may have reduced affinity for CD122 and/or CD132, and may comprise one or more mutations selected from L12G, L12K, L12Q, L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20F, D20G, D20T, D20W, M23R, R81A, R81G, R81S, R81T, D84A, D84E, D84G, D84I, D84M, D84Q D84R, D84S, D84T, S87R, N88A, N88D, N88E, N88F, N88G, N88M, N88R, N88S, N88V, N88W, N90T, N90S, V91D, V91E, V91G, V91S, 192K, I92R, I92T, 192S, E95G, Q126E, Q126F, Q126G, Q126I, Q126L, Q126M, Q126N, Q126R, Q126V, and Q126Y. Unless otherwise indicated, all residue numbers in IL-2 are in accordance with the numbering of SEQ ID NO: 1. In some embodiments, the IL-2 mutein may have mutations that result in enhanced affinity for CD25. Such mutations may be selected from mutations at positions 69 and 74. In some embodiments, the IL-2 mutein may comprise one or more mutations selected from T3A, C125A, C125S, and C125G.

B. Masking Moieties of the Isolated IL-2 Fusion Molecules

The cytokine antagonist, i.e., the masking moiety, in the present isolated IL-2 fusion molecule is an IL-2Rβ or IL-2Rγ extracellular domain or its functional analog such as one derived from human IL-2Rβ or IL-2Rγ (e.g., one of SEQ ID NOs: 3-6). In some embodiments, the IL-2 fusion molecule comprises at least one masking moiety. For example, the fusion molecule may comprise both an IL-2Rβ ECD and an IL-2Rγ ECD or just one of these ECDs. The ECD may comprise the entirety of the extracellular domain of human IL-2Rβ or IL-2Rγ, or contain only a portion thereof, so long as the portion remains able to bind to the IL-2 moiety or otherwise inhibiting the IL-2 moiety from binding to IL-2Rβ or IL-2Rγ on T cells.

In some embodiments, the IL-2 fusion molecule comprises a further masking moiety that is an ECD of IL-2Rα (e.g., SEQ ID NO: 7) or a functional analog thereof, wherein the IL-2Rα ECD masking moiety is fused to the cytokine moiety, the carrier moiety, or another masking moiety in the fusion molecule through a cleavable peptide linker. The presence of an IL-2Rα masking moiety linked to the fusion molecule via a cleavable linker allows the fusion molecule to home to a targeted site without binding to cells in non-targeted sites; once at the targeted site, the cleavable linker is cleaved by a protease present in high concentrations at the targeted site, allowing the activated fusion molecule to bind IL-2Rα on cells (e.g., Treg cells) at the targeted site and to stimulate the bound cells.

A functional analog of an ECD of an IL-2R subunit (α, β, or γ) refers to a polypeptide that has an affinity similar to that of the wildtype ECD for IL-2. For example, the functional analog contains the core IL-2 binding region of the wildtype ECD and may have a sequence that is at least 95% (e.g., at least 96, 97, 98, or 99%) identical to the wildtype ECD (e.g., SEQ ID NOs: 3-7, infra) across the entire length of the analog.

C. Carrier Moieties of the Isolated IL-2 Fusion Molecules

The carrier moieties of the present IL-2 fusion molecules may be an antigen-binding moiety, or a moiety that is not an antigen-binding moiety. The carrier moiety may improve the PK profiles such as serum half-life of the cytokine agonist polypeptide, and may also target the cytokine agonist polypeptide to a target site in the body, such as a tumor site.

1. Antigen-Binding Carrier Moieties

The carrier moiety may be an antibody or an antigen-binding fragment thereof, or an immunoadhesin. In some embodiments, the antigen-binding moiety is a full-length antibody with two heavy chains and two light chains, a Fab fragment, a Fab′ fragment, a F(ab′)₂ fragment, a Fv fragment, a disulfide linked Fv fragment, a single domain antibody, a nanobody, or a single-chain variable fragment (scFv). In some embodiments, the antigen-binding moiety is a bispecific antigen-binding moiety and can bind to two different antigens or two different epitopes on the same antigen. The antigen-binding moiety may provide additional and potentially synergetic therapeutic efficacy to the cytokine agonist polypeptide.

The IL-2 polypeptide and its mask may be fused to the N-terminus or C-terminus of the light chains and/or heavy chains of the antigen-binding moiety. By way of example, the IL-2 polypeptide and its mask may be fused to the antibody heavy chain or an antigen-binding fragment thereof or to the antibody light chain or an antigen-binding fragment thereof. In some embodiments, the IL-2 polypeptide is fused to the C-terminus of one or both of the heavy chains of an antibody, and the cytokine's mask is fused to the other terminus of the cytokine moiety through a non-cleavable or cleavable peptide linker. In some embodiments, the IL-2 polypeptide is fused to the C-terminus of one of the heavy chains of an antibody, and the cytokine's mask is fused to the C-terminus of the other heavy chain of the antibody through a non-cleavable or cleavable peptide linker, wherein the two heavy chains contain mutations that allow the specific pairing of the two different heavy chains.

Strategies of forming heterodimers are well known (see, e.g., Spies et al., Mol Imm. (2015) 67(2)(A):95-106). For example, the two heavy chain polypeptides in the isolated IL-2 fusion molecule may form stable heterodimers through “knobs-into-holes” mutations. “Knobs-into-holes” mutations are made to promote the formation of the heterodimers of the antibody heavy chains and are commonly used to make bispecific antibodies (see, e.g., U.S. Pat. No. 8,642,745). For example, the Fc domain of the antibody may comprise a T366W mutation in the CH3 domain of the “knob chain” and T366S, L368A, and/or Y407V mutations in the CH3 domain of the “hole chain.” An additional interchain disulfide bridge between the CH3 domains can also be used, e.g., by introducing a Y349C mutation into the CH3 domain of the “knobs chain” and an E356C or S354C mutation into the CH3 domain of the “hole chain” (see, e.g., Merchant et al., Nature Biotech (1998) 16:677-81). In other embodiments, the antibody moiety may comprise Y349C and/or T366W mutations in one of the two CH3 domains, and E356C, T366S, L368A, and/or Y407V mutations in the other CH3 domain. In certain embodiments, the antibody moiety may comprise Y349C and/or T366W mutations in one of the two CH3 domains, and S354C (or E356C), T366S, L368A, and/or Y407V mutations in the other CH3 domain, with the additional Y349C mutation in one CH3 domain and the additional E356C or S354C mutation in the other CH3 domain, forming an interchain disulfide bridge (numbering always according to EU index of Kabat; Kabat et al., “Sequences of Proteins of Immunological Interest,” 5th ed., Public Health Service, National Institutes of Health, Bethesda, Md. (1991)). Other knobs-into-holes technologies, such as those described in EP1870459A1, can be used alternatively or additionally. Thus, another example of knobs-into-holes mutations for an antibody moiety is having R409D/K370E mutations in the CH3 domain of the “knob chain” and D399K/E357K mutations in the CH3 domain of the “hole chain” (EU numbering).

In some embodiments, the antibody moiety in the isolated IL-2 fusion molecule comprises L234A and L235A (“LALA”) mutations in its Fc domain. The LALA mutations eliminate complement binding and fixation as well as Fcγ dependent ADCC (see, e.g., Hezareh et al. J. Virol. (2001) 75(24):12161-8). In further embodiments, the LALA mutations are present in the antibody moiety in addition to the knobs-into-holes mutations.

In some embodiments, the antibody moiety comprises the M252Y/S254T/T256E (“YTE”) mutations in the Fc domain. The YTE mutations allow the simultaneous modulation of serum half-life, tissue distribution and activity of IgG₁ (see Dall'Acqua et al., J Biol Chem. (2006) 281(33): 23514-24; and Robbie et al., Antimicrob Agents Chemother. (2013) 57(12):6147-53). In further embodiments, the YTE mutations are present in the antibody moiety in addition to the knobs-into-holes mutations. In particular embodiments, the antibody moiety has YTE, LALA and knobs-into-holes mutations or any combination thereof.

In some embodiments, the antigen-binding moiety binds to IL-1β, IL-1β receptor, IL-4, IL-4 receptor, IL-6, IL-6 receptor, IL-13, IL-13 receptor, IL-17, IL-17 receptor, IL-23, IL-23 receptor, TNFα, or TNFα receptor.

2. Other Carrier Moieties

Other non-antigen-binding carrier moieties may be used for the present isolated IL-2 fusion molecules. For example, an antibody Fc domain (e.g., a human IgG₁, IgG₂, IgG₃, or IgG₄ Fc), a polymer (e.g., PEG), an albumin (e.g., a human albumin) or a fragment thereof, or a nanoparticle can be used.

By way of example, the IL-2 polypeptide and its antagonist may be fused to an antibody Fc domain, forming an Fc fusion protein. In some embodiments, the IL-2 polypeptide is fused (directly or through a peptide linker) to the C-terminus or N-terminus of one of the Fc domain polypeptide chains, and the cytokine mask is fused to the C-terminus or N-terminus of the other Fc domain polypeptide chain through a non-cleavable or cleavable peptide linker, wherein the two Fc domain polypeptide chains contain mutations that allow the specific pairing of the two different Fc chains. In some embodiments, the Fc domain comprises the holes-into-holes mutations described above. In further embodiments, the Fc domain may comprise also the YTE and/or LALA mutations described above. In some embodiment, the Fc domain comprises a mutation at N297 (EU numbering).

The carrier moiety of the isolated IL-2 fusion molecule may comprise an albumin (e.g., human serum albumin) or a fragment thereof. In some embodiments, the albumin or albumin fragment is about 85% or more, about 90% or more, about 91% or more, about 92% or more, about 93% or more, about 94% or more, about 95% or more, about 96% or more, about 97% or more, about 98% or more, about 99% or more, about 99.5% or more, or about 99.8% or more identical to human serum albumin or a fragment thereof.

In some embodiments, the carrier moiety comprises an albumin fragment (e.g., a human serum albumin fragment) that is about 10 or more, 20 or more, 30 or more 40 or more, 50 or more, 60 or more, 70 or more, 80 or more, 90 or more, 100 or more, 120 or more, 140 or more, 160 or more, 180 or more, 200 or more, 250 or more, 300 or more, 350 or more, 400 or more, 450 or more, 500 or more, or 550 or more amino acids in length. In some embodiments, the albumin fragment is between about 10 amino acids and about 584 amino acids in length (such as between about 10 and about 20, about 20 and about 40, about 40 and about 80, about 80 and about 160, about 160 and about 250, about 250 and about 350, about 350 and about 450, or about 450 and about 550 amino acids in length). In some embodiments, the albumin fragment includes the Sudlow I domain or a fragment thereof, or the Sudlow II domain or the fragment thereof.

D. Linker Components of the Isolated Fusion Molecules

The IL-2 polypeptide may be fused to the carrier moiety with or without a peptide linker. The peptide linker may be cleavable or non-cleavable. In some embodiments, the cytokine moiety is fused to the carrier through a peptide linker, wherein said peptide linker is selected from SEQ ID NOs: 40-46 and 55-57. In particular embodiments, the peptide linker comprises the amino acid sequence of SEQ ID NO: 42, 44, 45, 46, 55, 56, or 57.

The masking moiety may be fused to the cytokine moiety or to the carrier through a non-cleavable or cleavable linker or without a peptide linker. The cleavable linker may contain one or more (e.g., two or three) cleavable moieties (CM). Each CM may be a substrate for an enzyme or protease selected from legumain, plasmin, TMPRSS-3/4, MMP-2, MMP-9, MT1-MMP, cathepsin, caspase, human neutrophil elastase, beta-secretase, uPA, and PSA. In some embodiments, the masking moiety is fused to the carrier through a peptide linker, wherein said peptide linker is selected from SEQ ID NOs: 40-46, 55, 56, and 57. In particular embodiments, the peptide linker comprises the amino acid sequence of SEQ ID NO: 42, 44, 45, 46, 55, 56, or 67. In some embodiment, said peptide linker comprises at least 10 amino acids, 12 amino acids, 14 amino acids, 16 amino acids, 17 amino acids, 18 amino acids, 19 amino acids, 20 amino acids, 21 amino acids, 22 amino acids, 25 amino acids, 27 amino acids, or 30 amino acids.

Specific, nonlimiting examples of IL-2 polypeptides, cytokine masks, carriers, peptide linkers, and isolated IL-2 fusion molecules are shown in the Sequences section below. Further, the isolated fusion molecules of the present disclosure may be made by well-known recombinant technology. For examples, one more expression vectors comprising the coding sequences for the polypeptide chains of the isolated fusion molecules may be transfected into mammalian host cells (e.g., CHO cells), and cells are cultured under conditions that allow the expression of the coding sequences and the assembly of the expressed polypeptides into the isolated IL-2 fusion molecule complex.

### Pharmaceutical Compositions

Pharmaceutical compositions comprising the isolated IL-2 fusion molecules (i.e., the active pharmaceutical ingredient or API) of the present disclosure may be prepared by mixing the API having the desired degree of purity with one or more optional pharmaceutically acceptable excipients (see, e.g., Remington's Pharmaceutical Sciences, 16th Edition., Osol, A. Ed. (1980)) in the form of lyophilized formulations or aqueous solutions. Pharmaceutically acceptable excipients (or carriers) are generally nontoxic to recipients at the dosages and concentrations employed, and include, but are not limited to: buffers containing, for example, phosphate, citrate, succinate, histidine, acetate, or another inorganic or organic acid or salt thereof; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including sucrose, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, and acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The choice of pharmaceutical carrier, excipient or diluent may be selected with regard to the intended route of administration and standard pharmaceutical practice. Pharmaceutical compositions may additionally comprise any suitable binder(s), lubricant(s), suspending agent(s), coating agent(s) or solubilizing agent(s).

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route.

In some embodiments, the pharmaceutical composition of the present disclosure is a lyophilized protein formulation. In other embodiments, the pharmaceutical composition may be an aqueous liquid formulation.

### Methods of Treatment

The IL-2 fusion molecules can be used to treat an inflammatory or autoimmune disease. In some embodiments, a method of treating a disease (such an autoimmune disease) in a subject comprises administering to the subject an effective amount of an isolated IL-2 fusion molecule disclosed herein. In some embodiments, the inflammatory or autoimmune disease is selected from the group consisting of asthma, diabetes (e.g., Type I diabetes or latent autoimmune diabetes), lupus (e.g., systemic lupus erythematosus), arthritis (e.g., rheumatoid arthritis), allergy, organ graft rejection, GVHD, Addison's disease, ankylosing spondylitis, anti-glomerular basement membrane disease, autoimmune hepatitis, dermatitis, Goodpasture's syndrome, granulomatosis with polyangiitis, Graves' disease, Guillain-Barre syndrome, Hashimoto's thyroiditis, hemolytic anemia, Henoch-Schonlein purpura (HSP), juvenile myositis, Kawasaki disease, inflammatory bowel diseases (such as Crohn's disease and ulcerative colitis), multiple sclerosis, myasthenia gravis, neuromyelitis optica, PANDAS, psoriasis, psoriatic arthritis, Sjögren's syndrome, systemic scleroderma, systemic sclerosis, thrombocytopenic purpura, uveitis, vasculitis, vitiligo, and Vogt-Koyanagi-Harada Disease.

Generally, dosages and routes of administration of the present pharmaceutical compositions are determined according to the size and conditions of the subject, according to standard pharmaceutical practice. In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, intracranially, or intraspinally. In some embodiments, the composition is administered to a subject intravenously.

In some embodiments, the dosage of the pharmaceutical composition is a single dose or a repeated dose. In some embodiments, the doses are given to a subject once per day, twice per day, three times per day, or four or more times per day. In some embodiments, about 1 or more (such as about 2, 3, 4, 5, 6, or 7 or more) doses are given in a week. In some embodiments, the pharmaceutical composition is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2, 3, 4, 5, 7, 10, 15, or 20 or more doses).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art.

### EXEMPLARY EMBODIMENTS

Further particular embodiments of the present disclosure are described as follows. These embodiments are intended to illustrate the compositions and methods described in the present disclosure and are not intended to limit the scope of the present disclosure.

1. A mutant of IL-2Rβ-ECD, which comprises one or more point mutations, wherein said IL-2Rβ-ECD mutant has enhanced thermo stability compared to the wild type one.
2. The IL-2Rβ-ECD mutant of embodiment 1, which comprises one or more mutations at position(s) selected from 41-5 (deletion of the first five amino acids), F11, V21, L28, W38, L51, P52, V53, 163, P67, 177, V88, V92, M93, I95, M107, I110, V115, P156, L157, Q162, Q164, W166, P174, L187, F191, P196, P200, P207, W90, H150, W152, W166, W194, and W197 (numbering according to SEQ ID NO: 3).
3. The IL-2Rβ-ECD mutant of embodiment 1, which comprises mutations at sites selected from the following groups (numbering according to SEQ ID NO: 3)
a. F11 and F191;
b. L51, P52, and V53;
c. V92, M93, I95;
d. M107, P196, I110; and
e. P156 and L157
4. The IL-2Rβ-ECD mutant of embodiment 2 or 3, wherein said hydrophobic amino acid or amino acids are mutated to a hydrophilic amino acid or amino acids, selected from S, G, N, T, and Q.
5. The IL-2Rβ-ECD mutant of embodiment 3, which comprises mutations selected from the following groups (numbering according to SEQ ID NO: 3)
a. F11S and F191G;
b. L51S, P52G, and V53S;
c. V92S, M93G, I95G;
d. M107G, P196S, 10G; and
e. P156S and L157G
f. W166N
g. Q164E
h. W166N, V115S
i. W152N
j. W152N, W166N
k. V92S
l. W166N, V92S
m. L157S
n. W165N, W157S
6. The IL-2Rβ-ECD mutant of embodiment 1, which comprises an amino acid sequence selected from SEQ ID NO: 47, 48, and 49.
7. An isolated IL-2 fusion molecule which is useful for treating inflammatory and autoimmune diseases, comprising a cytokine moiety and a masking moiety, wherein said cytokine moiety comprises an IL-2 polypeptide or an IL-2 mutein, and said masking moiety comprises the extracellular domain (ECD) of IL-2Rβ or its functional analog or mutant; and wherein said fusion molecule preferentially stimulates T regulatory cells relative to other T cells or NK cells in an in vitro assay.
8. The isolated IL-2 fusion molecule of embodiment 7, wherein said fusion molecule has an EC50 value of less than about 1 nM in a CTLL-2 cell proliferation assay.
9. The isolated IL-2 fusion molecule of embodiment 7, wherein said fusion molecule has an EC50 value of less than about 0.1 nM in a CTLL-2 cell proliferation assay.
10. The isolated fusion molecule of any of embodiments 7-9, wherein said masking moiety comprises a mutant of IL-2Rβ-ECD of any of embodiments 1-6.
11. The isolated fusion molecule of any of embodiments 7-10, which further comprises the extracellular domain (ECD) of IL-2Rγ or its functional analog.
12. The isolated fusion molecule of any of embodiments 7-11, which further comprises a carrier.
13. The isolated fusion molecule of embodiment 12, wherein said masking moiety is linked to the carrier moiety through a cleavable or non-cleavable peptide linker.
14. The isolated fusion molecule of any of embodiments 7-13, wherein said IL-2 polypeptide or IL-2 mutein comprises a sequence of amino acids at least 95% identical to SEQ ID NO:1; and wherein said IL-2Rβ ECD or its functional analog or mutant has an amino acid sequence at least 95% identical to SEQ ID NO:3.
15. The isolated fusion molecule of any of embodiments 7-13, wherein said IL-2 mutein has an amino acid sequence as shown in SEQ ID NO: 2.
16. The isolated fusion molecule of any of embodiments 7-13, wherein said IL-2 mutein has at least one mutation selected from L12G, L12K, L12Q, L12S, Q.13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20F, D20G, D20T, D20W, M23R, R81A, R81G, R81S, R81T, D84A, D84E, D84G, D84I, D84M, D84Q D84R, D84S, D84T, S87R, N88A, N88D, N88E, N88F, N88G, N88M, N88R, N88S, N88V, N88W, N90T, N90S, V91D, V91E, V91G, V91S, I92K, I92R, I92T, I92S, E95G, Q126E, Q126F, Q126G, Q126I, Q126L, Q126M, Q126N, Q126R, Q126V, and Q126Y (numbering according to SEQ ID NO: 1).
17. The isolated fusion molecule of any of embodiments 7-16, wherein said carrier moiety is selected from a carrier moiety is a PEG molecule, an albumin, an albumin fragment, an antibody Fc domain, or an antibody or an antigen-binding fragment thereof.
18. The isolated fusion molecule of embodiment 17, wherein the carrier moiety comprises an antibody Fc domain with a mutation at N297 and/or mutations L234A and L235A (“LALA”) (EU numbering).
19. The isolated fusion molecule of embodiment 17 or 18, wherein the carrier moiety comprises an antibody Fc domain comprising knobs-into-holes mutations, and wherein the cytokine moiety and the masking moiety are fused to different polypeptide chains of the antibody Fc domain.
20. The isolated fusion molecule of embodiment 19, wherein the cytokine moiety and the masking moiety are fused to the C-termini of the two different polypeptide chains of the Fc domain or to the C-termini of the two different heavy chains of the antibody.
21. The isolated fusion molecule of claim 19, wherein the carrier is an antibody Fc domain, and wherein the cytokine moiety and the masking moiety are fused to the N-termini of the two different polypeptide chains of the Fc domain.
22. The isolated fusion molecule of embodiment 12, wherein the carrier moiety is an antibody Fc domain, and wherein it comprises a first polypeptide chain comprising a molecular formula selected from F1-L1-E1, F1-L1-E1-L2-E2, and F1-L1-E2-L2-E1, and a second polypeptide chain comprising a molecular formula F2-L3-C, wherein said F1 and F2 are the subunits of the Fc domain which form heterodimer, L1, L2 and L3 are peptide linkers, E1 is IL-2Rβ ECD or its functional analog, and E2 is IL-2Rγ ECD or its functional analog, and C is the cytokine moiety.
23. The isolated fusion molecule of embodiment 12, wherein the carrier moiety is an antibody Fc domain, and wherein it comprises a first polypeptide chain comprising a molecular formula selected from E1-L1-F1, E1-L1-E2-L2-F1, and E2-L1-E1-L2-F1, and a second polypeptide chain comprising a molecular formula C-L3-F2, wherein said F1 and F2 are the subunits of the Fc domain which form heterodimer, L1, L2 and L3 are peptide linkers, E1 is IL-2Rβ ECD or its functional analog, and E2 is IL-2Rγ ECD or its functional analog, and C is the cytokine moiety.
24. The isolated fusion molecule of embodiment 12, wherein the carrier moiety is an antibody Fc domain, and wherein it comprises a first polypeptide chain and a second polypeptide chain comprising molecular formulas selected from the following pairs:
a. F1-L1-E1 and F2-L2-C-L3-E2;
b. F1-L1-E1 and F2-L2-E2-L3-C;
c. F1-L1-E2 and F2-L2-C-L3-E1;
d. F1-L1-E2 and F2-L2-E1-L3-C;
e. E1-L1-F1 and E2-L2-C-L3-F2;
f. E1-L1-F1 and C-L2-E2-L3-F2;
g. E2-L1-F1 and E2-L2-C-L3-F2; and
h. E2-L1-F1 and C-L2-E1-L3-F2;
wherein said F1 and F2 are the subunits of the Fc domain which form heterodimer, L1, L2 and L3 are peptide linkers, E1 is IL-2Rβ ECD or its functional analog, and E2 is IL-2Rγ ECD or its functional analog, and C is the cytokine moiety.
25. The isolated fusion molecule of any of embodiments 22-24, wherein said IL-2Rβ ECD has an amino acid sequence at least 95% identical as that of SEQ ID NO: 3, said IL-2Rγ ECD has an amino acid sequence as shown in SEQ ID NO: 6, and said cytokine moiety comprises an IL-2 mutein with an amino acid sequence at least 95% identical as that of SEQ ID NO: 2.
26. The isolated fusion molecule of any of embodiments 21-25, wherein the Fc domain comprising knobs-into-holes mutations.
27. The isolated fusion molecule of any one of embodiments 18-26, wherein the knobs-into-holes mutations comprise a T366Y “knob” mutation on a polypeptide chain of the Fc domain, and a Y407T “hole” mutation in the other polypeptide of the Fc domain (EU numbering).
28. The isolated fusion molecule of any one of embodiments 18-21 and 26, wherein the knobs-into-holes mutations comprise Y349C and/or T366W mutations in the CH3 domain of the “knob chain” and E356C, T366S, L368A, and/or Y407V mutations in the CH3 domain of the “hole chain” (EU numbering).
29. The isolated fusion molecule of embodiment 12, wherein the carrier moiety is an antibody Fc domain, and wherein the fusion molecule comprises a first polypeptide chain comprising an amino acid sequence at least 99% identical as one selected from SEQ ID NO: 8-11, 28, 29, and 30 and a second polypeptide chain with an amino acid sequence at least 99% identical as one selected from SEQ ID NO: 16-21.
30. The isolated fusion molecule of embodiment 12, wherein the carrier moiety is an antibody Fc domain, and wherein the fusion molecule comprises a first polypeptide chain comprising an amino acid sequence at least 99% identical as one selected from SEQ ID NO: 12-15, 31, 32, and 33 and a second polypeptide chain with an amino acid sequence at least 99% identical as one selected from SEQ ID NO: 22-27.
31. The isolated fusion molecule of embodiment 29 or 30, wherein said Fc domain further comprises a mutation of N297A or N297G (EU numbering).
32. The isolated fusion molecule of embodiment 12, wherein said carrier is an IgG4 Fc, which also comprises the knobs-into-holes mutations.
33. The isolated fusion molecule of embodiment 32, which comprises a first polypeptide chain comprising an amino acid sequence at least 99% identical or 100% identical as one selected from SEQ ID NO: 50, 51 and 52, and a second polypeptide chain with an amino acid sequence at least 99% identical or 100% identical as one selected from SEQ ID NOs: 53 and 54.
34. The isolated fusion molecule of any of embodiments 22-24, wherein said peptide linkers L1, L2, and L3 independently have an amino acid sequence selected from SEQ ID NOs: 40-46, 55-57, 59 and 60.
35. The isolated fusion molecule of any of embodiments 22-24, wherein at least one of the said peptide linkers L1, L2, and L3 has an amino acid sequence comprises 20-44 amino acids.
36. The isolated fusion molecule of any of embodiments 7-11, wherein said fusion molecule binds to the high affinity IL-2 receptor with alpha, beta and gamma subunits (IL-2Rαβγ) with at least 100 times stronger affinity than binds to the moderate affinity IL-2 receptor formed with the beta and gamma subunits (IL-2Rβγ).
37. The isolated fusion molecule of any of embodiments 7-11, which binds to IL-2Rβγ with a binding dissociation equilibrium constant (KD) of more than about 5 nM as measured in a surface plasmon resonance assay at 37° C.
38. The chimeric molecule according to any of embodiments 7-37, which promotes FOXP3-positive regulatory T cell growth or survival in vitro. 39. The chimeric molecule according to any of embodiments 7-37, which induces STATS phosphorylation in ex vivo FOXP3-positive T cells comprising a functional IL-2 receptor complex but has a reduced ability to induce phosphorylation of STATS in FOXP3-negative T cells.
40. The fusion molecule of any of embodiments 7-11, which further comprises the extracellular domain (ECD) of IL-2Rα or its functional analog; wherein said IL-2Rα ECD or its functional analog is linked to the fusion molecule through a cleavable peptide linker.
41. The fusion molecule of embodiment 40, said IL-2Rα ECD or its functional analog comprises an amino acid sequence at least 95% identical as the one shown in SEQ ID NO: 7.
42. A polynucleotide or polynucleotides encoding the fusion molecule of any one of embodiments 7-41 or the IL-2Rβ-ECD mutant of any of embodiments 1-6.
43. An expression vector or vectors comprising the polynucleotide or polynucleotides of embodiment 42.
44. A host cell comprising the vector(s) of embodiment 43.
45. A method of making the isolated fusion molecule of any one of embodiments 7-41, comprising culturing the host cell of claim 44 under conditions that allow expression of the fusion molecule, and isolating the fusion molecule.
46. A pharmaceutical composition comprising the chimeric molecule of any of embodiments 7-41 and a pharmaceutically acceptable excipient.
47. A method of treating an inflammatory or autoimmune disease in a subject, said method comprising administering to a subject in need thereof a therapeutically effective amount of a chimeric molecule of any of embodiments 7-41.
48. A method of treating an inflammatory or autoimmune disease in a subject, said method comprising administering to a subject in need thereof a therapeutically effective amount of the pharmaceutical composition of embodiment 46.
49. The method of claim 48, wherein the inflammatory or autoimmune disease is selected from the group consisting of asthma, diabetes, arthritis, allergy, organ graft rejection and graft-versus-host disease.

### EXAMPLES

### Transient Transfection

For transient transfection with HEK293 cells, expression plasmids were co-transfected into 3×10⁶ cells/mL freestyle HEK293 cells at 2.5-3 μg/mL using PEI (polyethylenimine). For Fc-based IL-2 isolated IL-2 fusion molecules, the ratios for the Fc-IL-2 mutein fusion polypeptide and the Fc-masking moiety fusion polypeptide were in a 1:2 ratio. For antibody-based IL-2 isolated IL-2 fusion molecules, ratios for the knob heavy chain (containing IL-2 agonist polypeptide) and hole heavy chain (containing the masking moiety) and the light chain DNA were in a 2:1:2 molar ratio. The cell cultures were harvested 6 days later after transfection by centrifugation at 9,000 rpm for 45 min followed by 0.22 μM filtration.

For transient transfection with ExpiCHO cells, expression plasmids were co-transfected into 6×10⁶ cells/mL ExpiCHO-S cells at 1-2 μg/mL using Expifectamine CHO Reagent. For 982 D1, the ratios for the knob heavy chain IL-2 mutein fusion polypeptide and the hole heavy chain (containing the β-masking moiety polypeptide) were in a 1:4 ratio. Similarly, for 982 D2, the ratios for the knob heavy chain IL-2E mutein polypeptide and the hole heavy chain (containing the β-masking moiety polypeptide) were in a 1:4 ratio. The cell cultures were harvested approximately 7 days later after transfection by centrifugation at 12,000 rpm for 40 min followed by 0.2 or 0.45 μM filtration.

### Protein Purification

The purifications of the proteins IL-2 fusion molecules (proteins) were carried out using Protein A affinity chromatography CaptivA® Resin (Repligen, Waltham, Mass.). For 982 Ref, 982 C1, and 982 C2 samples, further purifications were carried out using an anion exchange chromatography with Sepharose® Q FF resin or Sepharose® Q HP resin carried out in the flow-through mode, followed by a third column step using Capto™ MMC ImpRes resin. For 982 D1 and 982 D2 samples, further purifications were carried out using an anion exchange chromatography with Sepharose® Q HP resin carried out in the flow-through mode, followed by a third column step using Capto™ SP ImpRes resin. All the Sepharose® and Capto™ resins were ordered from GE Healthcare Life Sciences (now Cytiva, Marlborough, Mass.). The samples were purified to a purity of at least 98% by SEC-HPLC analysis prior to in vivo studies. The samples were formulated in 20 mM Histidine, 7% sucrose, 0.03% polysorbate-20. The samples were stored at −80° C. freezer until use.

### Proteolytic Treatment

Human MMP2 (Sino Biological #10082-HNAH) at 0.1 μg/μL was activated with 1 mM of p-aminophenylmercuric acetate (APMA, Sigma #A-9563). Two hundred (200) μg of the IL-2 fusion molecule was incubated with 0.5 μg of human MMP2 in the HBS buffer (20 mM HEPES, 150 mM NaCl2, pH7.4) containing 2 mM CaCl2 and 10 μM ZnCl2 at 37° C. for 16 hours (overnight).

### SDS-PAGE Analysis

Ten (10) μL of the culture supernatants or 20 μg of purified protein samples were mixed with Bolt™ LDS Sample Buffer (Novex) with or without reduce reagents. The samples were heated at 70° C. for 3 min and then loaded to a NuPAGE™ 4-12% BisTris Gel (Invitrogen). The gel was run in NuPAGE™ MOPS SDS Running buffer (Invitrogen) at 200 Volts for 40 min and then stained with Coomassie Blue.

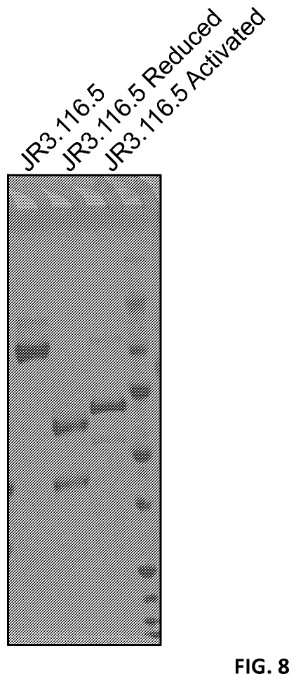

FIG. 8 shows the results of the SDS-PAGE analysis of the isolated IL-2 fusion molecule JR3.116.5 prior to activation (non-reduced and reduced) and post activation by the protease treatment as described above. JR3.116.5 comprises two polypeptide chains with amino acid sequence shown in SEQ ID NO: 12 and 23, respectively and has a structure as illustrated in FIG. 1B. The data indicated that the majority of the Protein A column pool was the intended heterodimer molecule of JR3.116.5. There appeared to be a small band of the homodimer of the hole chain (SEQ ID NO: 23). Surprisingly, there was no obvious band of unpaired chain or any homodimer of the knob chain. It is possible that the interaction between the cytokine moiety and the mask moiety promoted the correct heterodimerization between the knob chain (SEQ ID NO: 12) and the hole chain (SEQ ID NO: 23).

### CTLL-2 Assay

CTLL-2 cells were grown in the RPMI 1640 medium supplemented with L-glutamine, 10% fetal bovine serum, 10% non-essential amino acids, 10% sodium pyruvate, and 55 μM beta-mercaptoethanol. CTLL-2 cells were non-adherent and maintained at 5×10⁴-1×10⁶ cells/mL in medium with 100 ng/mL of IL-2. Generally, cells were split twice per week. For bioassays, it was best to use cells no less than 48 hours after passage.

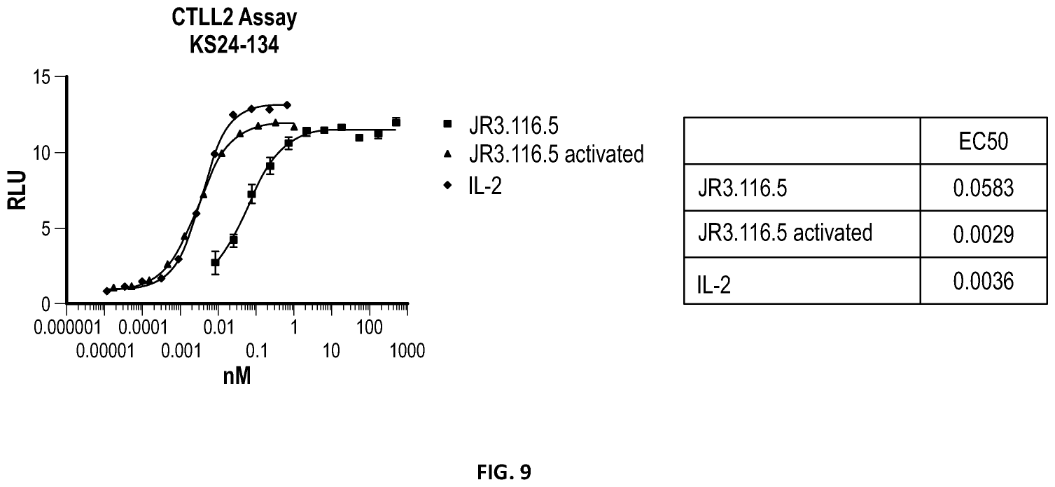

Samples were diluted at 2× concentration in 50 μL/well in 96-well plates. The IL-2 standards were titrated from 20 ng/mL (2× concentration) to 3× serial dilutions for 12 wells. Samples were titer tested as appropriate. CTLL-2 cells were washed 5 times to remove IL-2, 5000 cells/well were dispensed in 50 μL and cultured overnight or at least 18 hours with the samples. Subsequently, 100 μL/well Cell Titer Glo reagents (Promega) were added and luminescence was measured. FIG. 9 shows the results of the CTLL-2 analysis. The data indicated that the masking moiety reduced the activity of JR3.116.5 by approximately 20 folds. In addition, this masking effect was reversible as the activation by protease cleavage of the masking moiety restored the activity of the fusion molecule.

### NK92 Cell Proliferation Assay

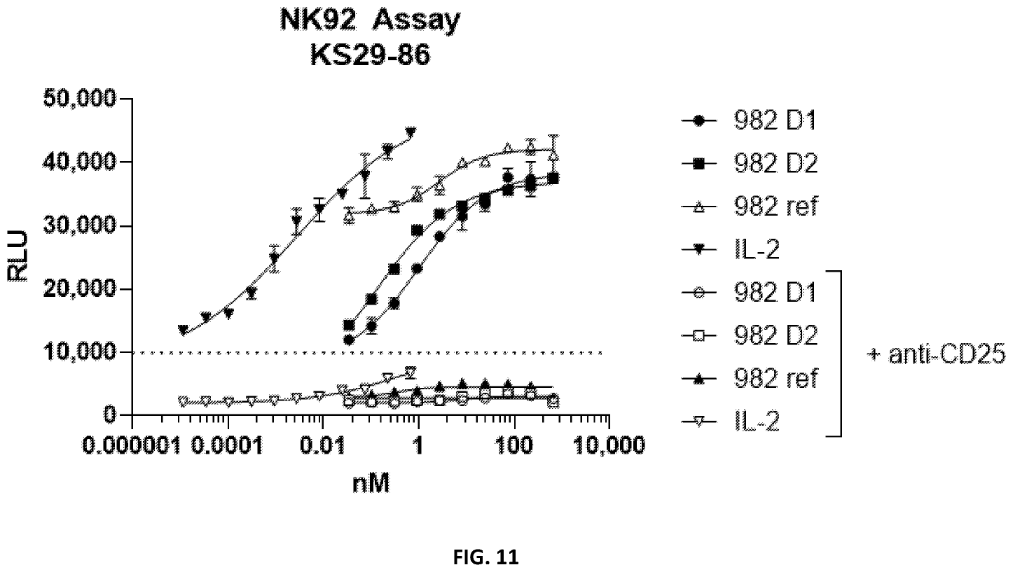

The NK92 cell line is a factor dependent cell line that requires IL-2 for growth and survival. Prior to the assay the NK92 cells were washed to remove 1L2 and cultured overnight without growth factor. Cells were harvested and washed again to remove residual growth factor. Cells were resuspended to 4000,000 cells/mL. Cells (20,000/well) were then added to 96-well plates. An anti-CD25 antibody, basiliximab, was added to half of the plate (48 wells) at 10 μg/mL. Cells were incubated for 15 min. Serial titrations of IL-2 fusion molecules were added to each well at 50 μL/well. Plates were incubated overnight, and Cell Titer Glo (Promega) was added prior to measuring luminescence. This provided a measure of ATP levels as an indicator of cell viability. FIG. 11 shows the NK92 proliferation assay of 982 D1, 982 D1, and 982 Ref with and without the presence of an anti-CD25 neutralizing antibody. The reference molecule (982 Ref) is an IL-2 fusion molecule with IL-2 having substitution mutations V91K and C125A (IL-2 moiety numbering according to SEQ ID NO: 1). For the assays with anti-CD25 antibody, the anti-CD25 antibody was added to the cells at 10 μg/mL.

The data indicated that 982 Ref had stronger activity than 982 D1 and 982 D2 in stimulating the proliferation of the NK92 cells. All of the tested fusion molecules showed minimum activities when neutralizing anti-CD25 antibody was added to the assay.

### Binding Assay: Rat CD4⁺ T Cells

Blood samples collected from Male Sprague-Dawley rats with jugular vein cannulas were lysed to remove red blood cells. The remaining cells were incubated with various concentrations of the test article 982 D1 or 982 D2 for approximately 60 min on ice. The detection antibody, goat anti-human IgG Fcγ-APC (Jackson ImmunoResearch Lab. Cat #109-135-170), was added to each well. After incubation and subsequent wash, an anti-rat CD4 antibody (BD Bioscience, cat #554866) was added to stain rat CD4 T cells. The stained samples were washed again and then subject to flow cytometry analysis for detection of IL-2 fusion molecules' binding to rat CD4⁺ T cells.

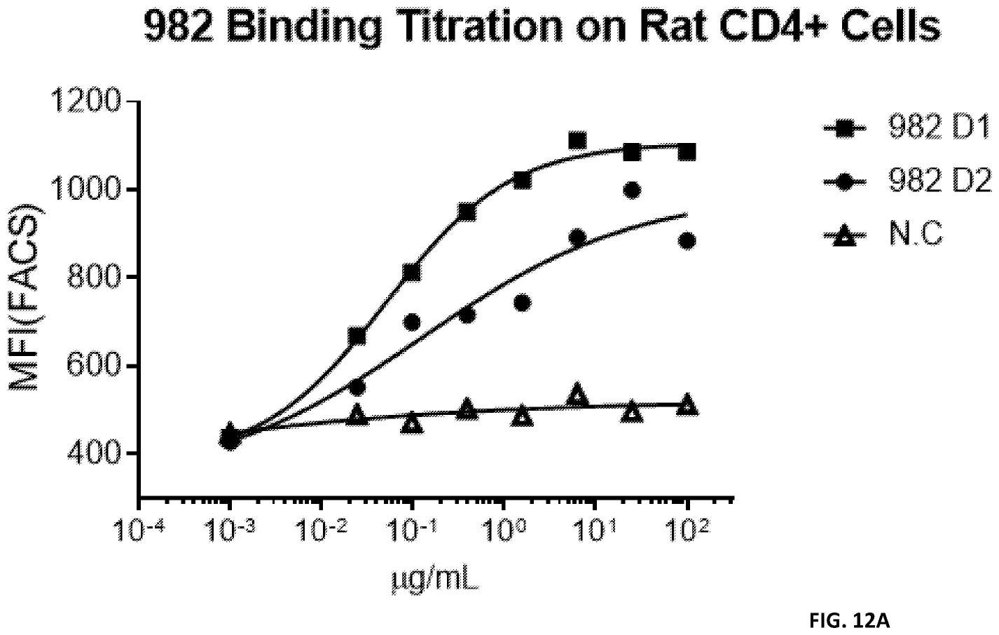

FIG. 12 shows the binding activity of 982 D1 and 982 D2 to rat CD4⁺ T cells. N.C. represents irrelevant Ab control. Surprisingly, 982 D1 showed a minimally stronger binding to the rat CD4+ T cells than 982 D2, though the difference was not significant.

Binding Assay: Human CD4⁺CD25⁺ T Cells

Human peripheral blood mononuclear cells (hPBMCs) were isolated from buffy coat blood (BioIVT and RBC) and were cultured in the complete medium RPMI 1640 (Life Technologies, cat #12633-020) containing 10% FBS (Life Technologies, cat #10099141) overnight. Next day human PBMCs were treated with an anti-human CD3 antibody (Biolegend cat #317302) for 2 days, washed 3 times with complete medium RPMI 1640, then rested for 3 days. The cells were adjusted to a concentration of 4-5×10⁶ cells/mL with the washing buffer, then 50 μL of cells (200-250K cells/well), followed by 50 μL of IL-2 fusion molecules 982 C1, 982 D1, 982 D2, and 982 Ref were loaded to corresponding wells of a 96-well plate at various concentrations. The irrelevant Ab was added at the same various concentration ranges as a negative control. After incubation for approximate 60 min on ice, the cells were washed, then the detection antibody, goat anti-human IgG Fcγ-APC, (Jackson ImmunoResearch Lab. Cat #109-135-170) was added. After extensive washing to remove free IgG Fcγ-APC, FITC-conjugated mouse anti-human CD4 Ab (BD bioscience, cat #555346) and PE-conjugated mouse anti-human CD25 Ab (BD bioscience, cat #555432) were added to wells for cell staining. Lastly the stained samples were subject to flow cytometry analysis for detection of IL-2 fusion molecules' binding to Treg (CD4⁺CD25+) and T_{eff} (CD4⁺CD25⁻) cells, respectively.

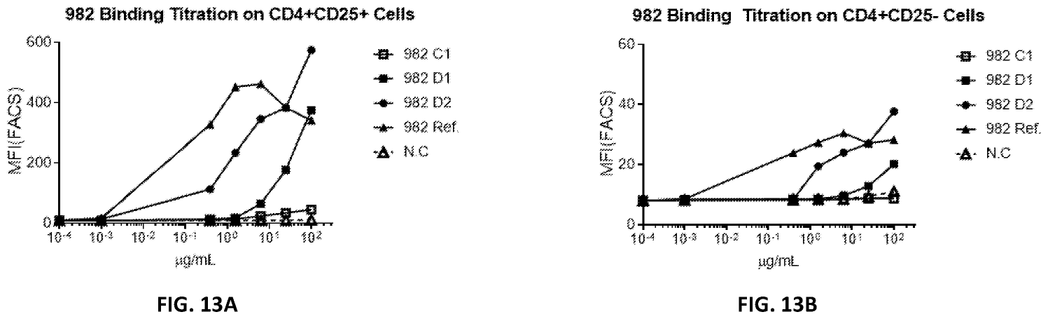

FIGS. 13A and 13B show the bindings of 982 C1, 982 D1, and 982 Ref to human CD4⁺/CD25⁺ T cells and CD4⁺/CD25⁻ T cells. The results showed that 982 Ref had stronger binding affinity to the CD4⁺/CD25⁺ T cells than that of 982 D2, 982 D1 and 982 C1. In this assay, PBMCs were treated with an anti-CD3 antibody for 2 days, rested for 3 days, and then were incubated with various concentrations of IL-2 fusion molecules 982 C1, 982 D1, 982 Ref, and a buffer control (N.C.) for approximately 40 minutes at room temperature. An anti-hFc secondary antibody was added, followed by anti-CD4 and anti-CD25 antibody staining. The stained samples were subject to flow cytometry analysis for detection of IL-2 fusion molecule binding on Treg (CD4⁺CD25⁺) and T_{eff} (CD4⁺CD25⁻) cells, respectively. Subsequent potency comparison among 982 C1, 982 D1, and 982 D2 showed binding potency in the rank order of 982 D2>982 D1>982 C1. The IL-2 moiety of 982 D2 comprises two point mutations that enhance its binding to CD25. These results suggested that masking with IL-2Rβ-ECD reduced the binding of 982 D1, while the double masking with IL-2Rβ-ECD and IL-2Rγ-ECD resulted in further reduced binding of the masked IL-2 fusion molecule 982 C1. Though the similar rank order of binding activity was observed in CD4⁺CD25⁻ T cells, the respective MFI for 982 Ref, 982 D1, 982 D2 and 982 C1 binding was relatively low as compared to that in CD4⁺CD25⁺ T cells, indicating the preferential binding of the IL-2 fusion molecules toward CD4⁺CD25⁺ T cells.

### T Cell Proliferation Assay

Human PBMCs isolated from buffy coat blood (BioIVT and RBC) were treated with anti-CD3 antibody (Biolegend cat #317302) for 2 days and then rested for 3 days. The cells were incubated with various concentrations of IL-2 fusion molecules 982 C1, 982 D1, 982 D2, or 982 Ref as indicated, or IL-2 for 3-days at 37° C., 5% CO₂ incubator. Then the cells were lysed/fixed/permeabilized, followed by antibody staining with mouse anti-human CD4-FITC (BD bioscience, cat #555346), mouse anti-human CD25-PE (BD bioscience, cat #555432), and mouse anti-human Ki67 Alex-647 (BD bioscience, cat #558615). After washing, stained cells were subject to flow cytometry analysis for Ki67+(proliferation marker) cells on Treg (CD4⁺CD25⁺) and T_{eff} (CD4⁺CD25⁻) cells, respectively.

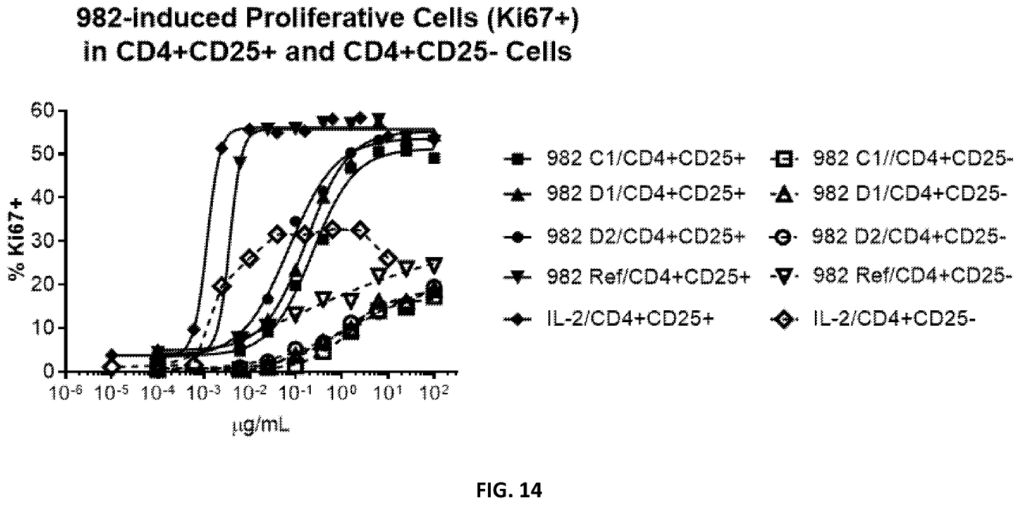

FIG. 14 shows the concentration-dependent proliferation of CD4⁺CD25⁺ T cells and CD4⁺CD25⁻ T cells induced by the 982 D1, 982 C1, 982 D2, and 982 Ref IL-2 fusions molecules. In this assay, PBMCs were treated with anti-CD3 antibody for 2 days and rested for 3 days. PBMCs were then incubated with various concentrations of IL-2 fusion molecules 982 C1, 982 D1, 982 D2, 982 Ref as indicated, or IL-2 for 3-days at 37° C., 5% CO₂ incubator. The cells were then lysed/fixed/permeabilized, and stained with anti-CD4, CD25, and Ki67 antibodies. After washing, stained cells were subjected to flow cytometry analysis for Ki67⁺ (proliferation marker) cells on Treg (CD4⁺CD25⁺) and Teff (CD4⁺CD25⁻) cells, respectively.

The in vitro activities of the IL-2 fusion molecules, from strongest to weakest, were in the following order: 982 Ref, 982 D2, 982 D1, and 982 C1 with overall much greater proliferation observed in CD4⁺CD25⁺ T cells than in CD4⁺CD25⁻ T cells. In summary, the results showed that 982 Ref had the strongest activities in all of three in vitro assays, followed by 982 D2, 982 D1, and 982 C1, in that order. These results are consistent with the binding activity results shown in FIG. 13.

### Rat PK and PD Study

Male Sprague-Dawley rats with jugular vein cannulas were dosed with IL-2 fusion molecules at 1 mg/kg or 3 mg/kg subcutaneously. Blood was sampled at various time points from 0-144 hours.

For PK analysis, serum samples were assayed for test article by ELISA. Briefly, ELISA plates were coated with 100 μL/well F(ab′)2 goat anti-human IgG Fcγ (Jackson ImmunoResearch, Cat. #109-006-170) at 2 μg/mL in PBS. Plates were incubated overnight at 4° C. The plates were blocked with 100 μL/well of PBS with 10% goat serum. After 1 hour of incubation and subsequent wash (four times with DI water), 100 μL of the serum samples diluted in PBS/10% goat serum or standard was added to each well. After incubation (1 hour) and wash (6 times with DI water), 100 μL of a 2^{nd} antibody (anti-IL2-biotin (R&D Systems BAF202) at 0.5 μg/mL in PBS/10% goat serum was added to each well. After incubation (1 hour) and wash (6 times with DI water), 100 μL of Streptavidin-HRP (Jackson ImmunoResearch, Cat. #016-30-84, 1:1000) in PBS/10% goat serum was added to each well. After incubation (1 hour) and wash (8 times with DI water). The color reaction was started by adding 100 μL of the TMB substrate to each well. The reaction was stopped with the addition of 100 μL/well of 1N H₂SO₄ solution. OD450 was then measured.

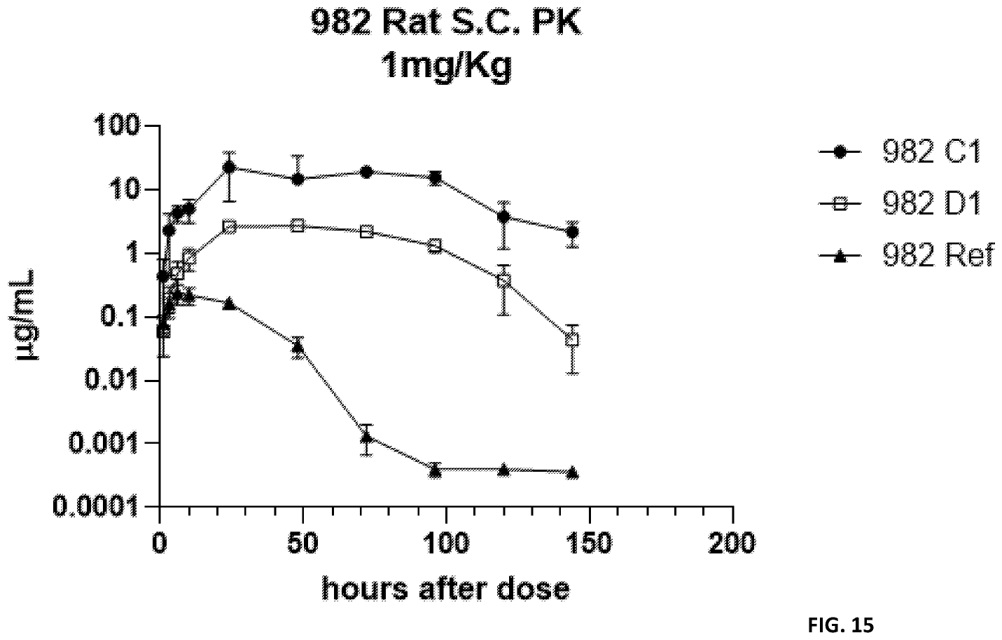

FIG. 15 showed the serum plasma concentration of 982 C1, 982 D1, and 982 Ref IL-2 fusion molecules over time from a rat PK study. In this assay, male Sprague-Dawley rats with jugular vein cannulas were dosed with IL-2 fusion molecules 982 C1, 982 D1, and 982 Ref at 1 mg/kg subcutaneously. Blood was sampled at 0, 1, 3, 6, 10, 24, 48, 72, 96, 120 and 144 hours. Serum samples were assayed for IL-2 fusion molecules by ELISA using goat anti-human IgG Fc gamma capture and anti-human IL-2 biotin as detection reagent. 982 C1 had greater AUC₍₀₋ₜ₎ (area under the concentration time curve up to the last measurable concentration) than 982 D1, while both had significantly greater AUC₍₀₋ₜ₎ than 982 Ref.

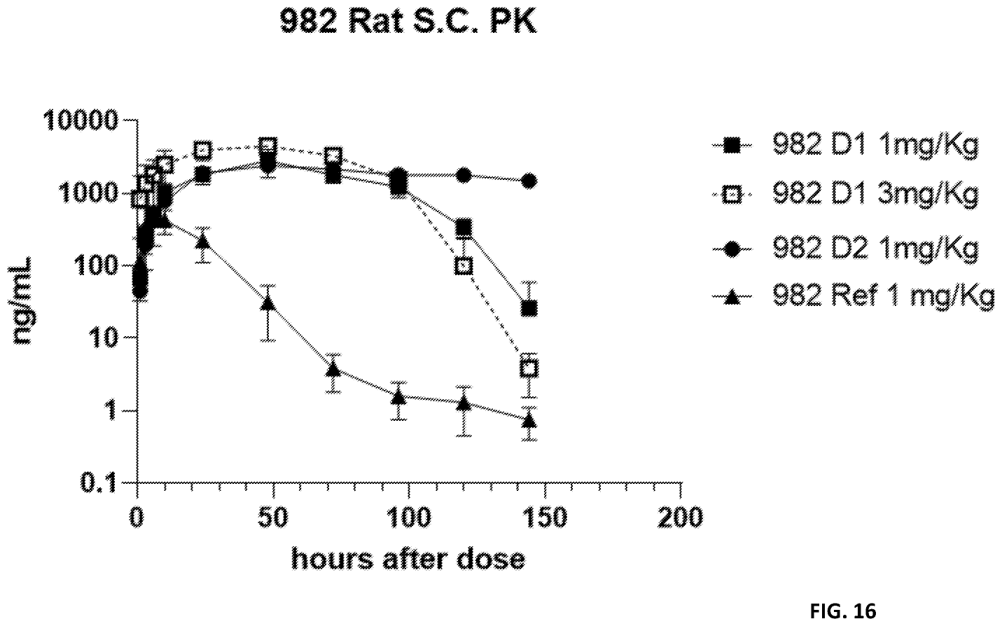

FIG. 16 showed the serum plasma concentration of 982 D1, 982 Ref, and 982 D2 IL-2 fusion molecules over time from a second rat study. In this assay, male Sprague-Dawley rats with jugular vein cannulas were dosed subcutaneously with 1 mg/kg of IL-2 fusion molecules 982 D1, 982 D2, and 982 Ref and 3 mg/kg of 982 D1, as indicated. Blood was sampled at 0, 1, 3, 6, 10, 24, 48, 72, 96, 120 and 144 hours. Serum samples were assayed for test article by ELISA using goat anti-human IgG Fc gamma capture and anti-human IL-2 biotin as detection reagent. 982 D2 had greater AUC(0-t) than 982 D1, while both had significantly greater AUC(0-t) than 982 Ref. The serum plasma concentration over time results showed that the masked IL-2 fusion molecules had better PK profiles than 982 Ref, which had a V91K mutation in its IL-2 moiety.

For PD analysis, blood was sampled at various time points between 0-144 hours following subcutaneous injection of 982 molecules. Blood samples collected into K2 EDTA blood collection tubes from rats treated with 982 IL-2 fusion molecules, were lysed and fixed with one volume of each blood sample mixed with the freshly made and pre-warmed BD Phosflow™ lyse/fix buffer (1×, BD Biosciences cat #558049), according to manufacturer's recommendation. Blood samples were then washed 2 times with PBS containing 2% FBS followed by permeabilization with cold permeabilization buffer II (BD Biosciences cat #558052, −20° C.) on ice for 30 min, according to manufacturer's instructions. The cells were then washed extensively 4 times with PBS containing 2% FBS, and the cell pellets were either stored at 4° C. or resuspended in staining buffer.

For FOXP3 and Ki67 measurements, an aliquot of 50 μL/well of the fixed/permeabilized rat blood cells (300 k-400K cells/well) described above from each sampling was added into 96-well working plates. Then 50 μL of Ab mixture containing mouse anti-rat CD4-FITC (Biolegend, cat #201505), mouse anti-rat CD25-PE (BD Bioscience, cat #554866), and mouse anti-rat FOXP3-APC (Biolegend, cat #320014); or mouse anti Ki67-APC (Biolegend, cat #320514) was added to each well and cells in the plates were incubated for 1 hour at room temperature. The plates were washed 2 times with FACS buffer and then subjected to flow cytometry analysis for Treg (CD4⁺FOXP3⁺) and Teff (CD4⁺FOXP3⁻) cells in % changes, respectively, over time. The plates were also subject to flow cytometry analysis for Ki67⁺ (proliferation marker) cells in % changes over time in gated Treg (CD4⁺CD25⁺) and T_{eff} (CD4⁺CD25⁻) cells, respectively.

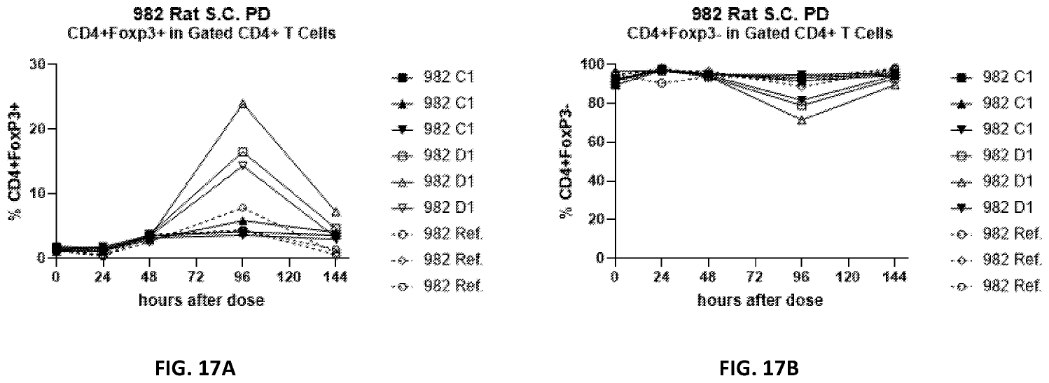

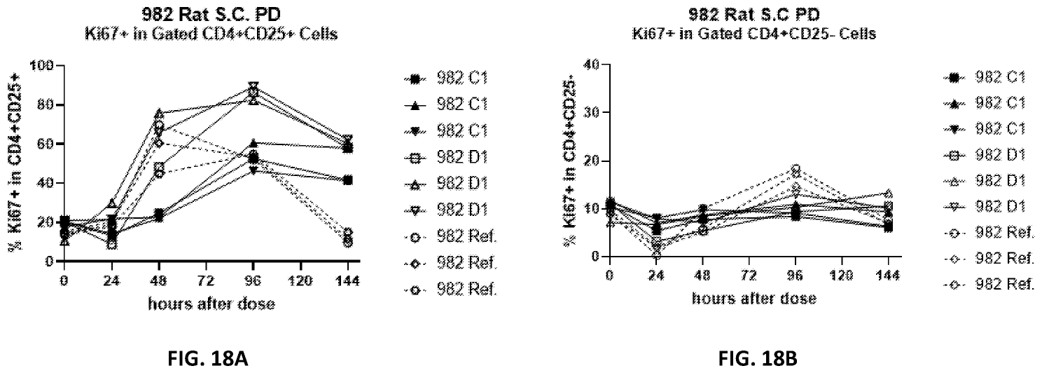

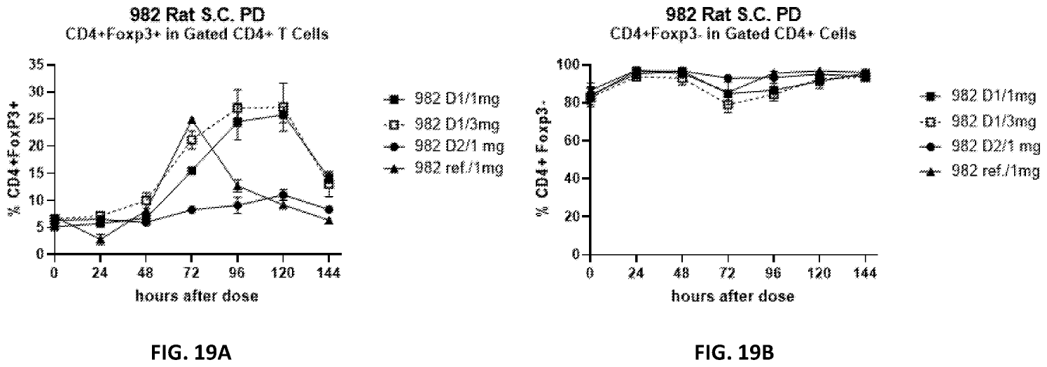

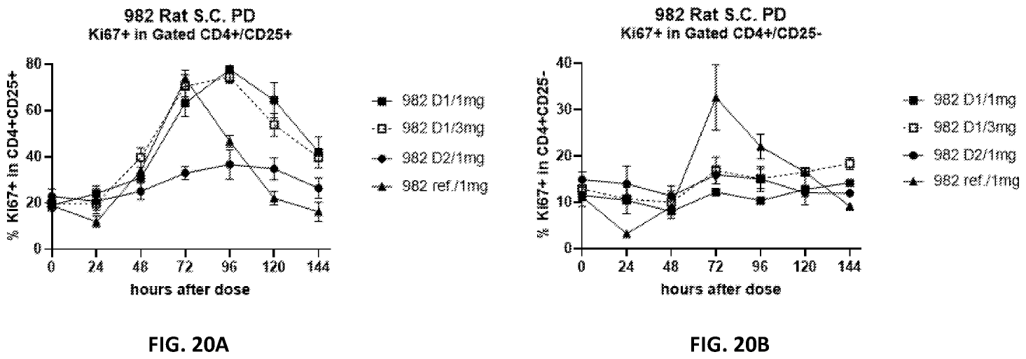

FIGS. 17A and 17B show changes induced in CD4⁺/FOXP3⁺ and CD4⁺/FOXP3⁻ cells (in rats) by 982 C1, 982 D1, and 982 Ref IL-2 fusion molecules. FIGS. 18A and 18B show proliferation of CD4⁺CD25⁺ and CD4⁺CD25⁻ cells induced by 982 C1, 982 D1, and 982 Ref IL-2 fusion molecules in rats from the first study. Male Sprague-Dawley rats with jugular vein cannulas were dosed with 1 mg/kg of IL-2 fusion molecules 982 C1, 982 D1 and 982 Ref subcutaneously. Blood was sampled at 0, 24, 48, 96 and 144 hours and were subject to Ab staining after blood samples lysis/fixation/permeabilization. This was followed by flow cytometry analysis for Treg (CD4⁺FOXP3⁺) and Teff (CD4⁺FOXP3⁻) cells in % changes, respectively, over time (FIGS. 17A and 17B or fir Ki67+ (proliferation marker) cells in % changes over time in gated Treg (CD4⁺CD25⁺) and T_{eff} (CD4⁺CD25⁻) cells, respectively, (FIGS. 18A and 18B). FIGS. 19A and 19B show the results of changes in CD4⁺/FOXP3⁺ and CD4⁺/FOXP3⁻ cells induced by the 982 IL-2 fusion molecules in rats. FIGS. 20A and 20B show the results of 982-IL-2 fusion molecule-induced proliferations of CD4⁺/CD25⁺ and CD4⁺/CD25⁻ cells in rats. Male Sprague-Dawley rats with jugular vein cannulas were dosed with IL-2 fusion molecules 982 D1 at 1 mg/kg and 3 mg/kg, 982 D2 at 1 mg/kg, and 982 Ref at 1 mg/kg subcutaneously. Blood was sampled at 0, 24, 48, 72, 96, 120 and 144 hours and were subject to Ab staining after blood samples lysis/fixation/permeabilization. This was followed by flow cytometry analysis for Treg (CD4⁺FOXP3⁺) and Teff (CD4⁺FOXP3⁻) cells in % changes, respectively, over time (FIGS. 19A and '9B) or by flow cytometry analysis for Ki67+ (proliferation marker) cells in % changes over time in gated Treg (CD4⁺CD25⁺) and T_{eff} (CD4⁺CD25⁻) cells, respectively (FIGS. 20A and 20B).

The results indicate that 982 D1 had greater and longer duration of effect on the CD4⁺FOXP3⁺ T cells and the CD4⁺CD25⁺ T cells than 982 Ref. This is surprising considering the significantly higher in vitro activities of 982 Ref were observed. Similar observations were made in the second in vivo rat study (FIGS. 19A, 19B, 20A, and 20B). Surprisingly, 982 D1 also demonstrated greater in vivo efficacy than that of 982 D2 (FIGS. 19A, 19B, 20A and 20B) in stimulating the proliferations of CD4⁺/FOXP3⁺ T cells and CD4⁺/CD25⁺ and CD4⁺/CD25⁻ cells in rats. This agrees well with the finding that 982 D1 showed slightly higher binding activity to rat CD4 T cells than 982 D2 in the binding assay (FIG. 12). However, the difference in the activities observed between 982 D1 and 982 D2 was more obvious in vivo than in vitro (FIGS. 19A and 20A).

While both 982 D1 and 982 Ref showed selectivity in preferentially stimulating Treg cells than T_{eff} cells, it was obvious that 982 D1 had better selectivity than 982 Ref as evident by little activity observed in stimulating the T_{eff} cells by 982 D1 as compared to 982 Ref (FIGS. 18B and 20B).

### Body Weights

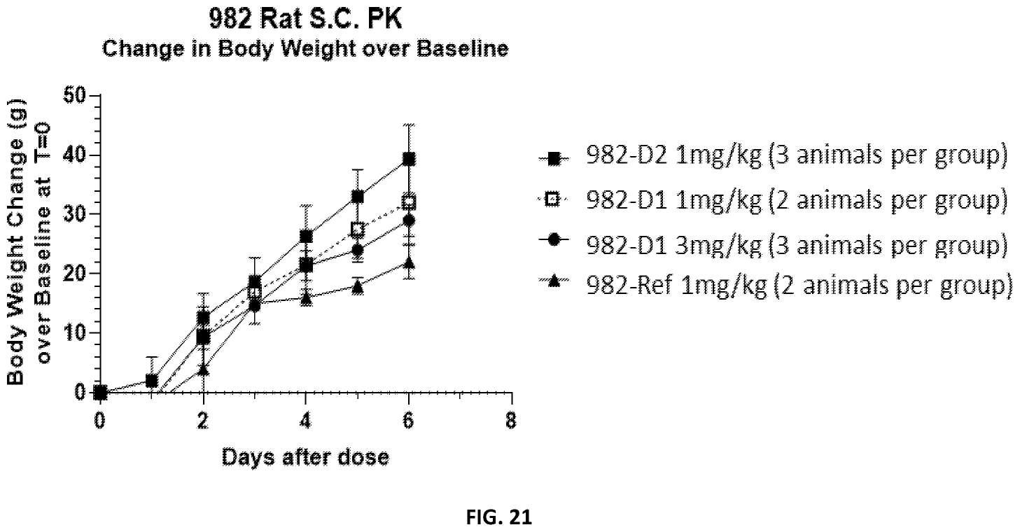

In order to assess the safety of the IL-2 fusion molecules, the body weight of the animals was also measured over the course of the 6-day study. Animals received a single subcutaneous administration of the IL-2 fusion molecules 982 D1 at 1 mg/kg and 3 mg/kg, 982 D2 at 1 mg/kg, and 982 Ref at 1 mg/kg. Body weight (BW) was measured for each animal daily between day 0 (dosing) and day 6. The results are shown in FIG. 21. The data demonstrated that rats receiving a single injection of 982 D1 at 1 mg/kg and at 3 mg/kg gained more body weight than rats receiving 982 Ref at 1 mg/kg.

In summary, the in vitro and in vivo studies described above demonstrated that the masked IL-2 fusion molecule 982 D1 had surprisingly better PK profiles than 982 Ref, which is a homodimer IL-2 fusion molecule comprising mutations V91K and C125A in its IL-2 moiety. Surprisingly, 982 D1 had more potent in vivo activity in stimulating the proliferation of CD4⁺CD25⁺ T cells and CD4⁺FOXP3⁺ T cells in rats than that of 982 Ref (FIGS. 17A, 17B, 18A, 18B, 19A, 19B, 20A, and 20B). In addition, all of the three masked IL-2 fusion molecules (982 C1, 982 D1, and 982 D2) had longer PKs than that of 982 Ref (FIGS. 15 and 16). It is also surprising that 982 D1 had stronger in vivo activity in rats than that of 982 D2. 982 D1 also had superior in vivo activity compared to the other molecules tested in the same rat studies, despite that it had relatively modest activity in vitro compared to 982 D2 and 982 Ref. In addition, the body weight data (FIG. 21) suggests that 982 D1 may potentially be safer as well than 982 Ref. It was also surprising that the potent and selective in vivo activity of the masked IL-2 fusion molecule 982 D1 can be achieved without the requirement of protease-dependent cleavage and removal of the masking moiety since 982 D1 does not comprise any cleavable peptide linker. This novel mode of action is desirable because the distribution of the protease(s) at the disease site(s) may not be even, and non-specific cleavage and removal (or “leaking”) of the masking moiety may take place in circulation or other normal tissues and outside of the disease sites.

Without wishing to be bound by theory, it is possible that the difference in PK profiles could partially explain the superior in vivo activities of 982 D1 comparing to that of 982 Ref. The species cross-reactivity could in part explain the observed difference in in vivo activities between 982 D1 and D2. Without wishing to be bound by theory, it is also possible that upon binding of the fusion molecule to CD25, the long linker between the masking moiety and the carrier in 982 D1 facilitates the competition of the endogenous IL-2Rβ ECD with the masking moiety when both endogenous IL-2Rα and IL-2Rγ are present. Binding of the cytokine moiety to both endogenous IL-2Rβ and IL-2Rγ is necessary for 982 D1 to stimulate the expansion of the Treg cells. The long linker between the masking moiety IL-2Rβ-ECD and the carrier may provide the flexibility needed for the cytokine moiety to form the tetrameric complex with the endogenous IL-2Rα, IL-2Rβ and IL-2Rγ. If the linker between the masking moiety IL-2Rβ-ECD and the carrier is short, and especially if the linker between the cytokine moiety and the carrier is also short, it would be possible that the masking moiety becomes a special constrain for the formation of the tetrameric complex. FIG. 11 showed that 982 D2 had slightly weaker binding to rat CD4⁺ T cells than that of D1, though 982 D2 had stronger in vitro activities with human T cells than that of 982 D1. However, the difference in the binding to rat CD4⁺ cells was relatively modest and may not explain the significant difference in the in vivo activities between D1 and D2 (19A, 19B, 20A, and 20B).

### Sequences

In the sequences below, boxed residues indicate mutations. Underlines in cleavable linkers indicate protease substrate sequences.

human IL-2
SEQ ID NO: 1
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
human IL-2 mutein
SEQ ID NO: 2

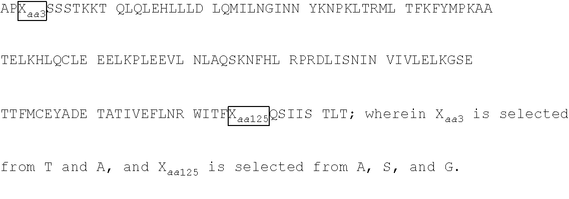

Human IL-2 Receptor Beta Subunit Extracellular
Domain (https://www.uniprot.org/uniprot/P14784)
SEQ ID NO: 3
AVNGTSQFTC FYNSRANISC VWSQDGALQD TSCQVHAWPD RRRWNQTCEL
LPVSQASWAC NLILGAPDSQ KLTTVDIVTL RVLCREGVRW RVMAIQDFKP
FENLRLMAPI SLQVVHVETH RCNISWEISQ ASHYFERHLE FEARTLSPGH
TWEEAPLLTL KQKQEWICLE TLTPDTQYEF QVRVKPLQGE FTTWSPWSQP
LAFRTKPAAL GKDT
Human IL-2 Receptor Beta Subunit Extracellular
Domain Mutant D68E (https://www.uniprot.org/uniprot/P14784)
SEQ ID NO: 4

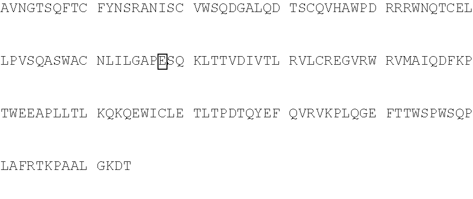

Human IL-2 Receptor Beta Subunit Extracellular
Domain Mutant E136Q/H138R
(https://www.uniprot.org/uniprot/P14784)
SEQ ID NO: 5

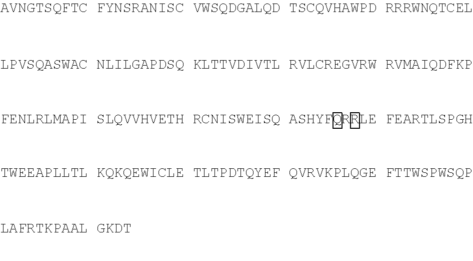

Human IL-2 Receptor Gamma Subunit Extracellular
Domain (http://www.uniprot.org/uniprot/P31785)
SEQ ID NO: 6
LNTTILTPNG NEDTTADFFL TTMPTDSLSV STLPLPEVQC FVFNVEYMNC
TWNSSSEPQP TNLTLHYWYK NSDNDKVQKC SHYLFSEEIT SGCQLQKKEI
HLYQTFVVQL QDPREPRRQA TQMLKLQNLV IPWAPENLTL HKLSESQLEL
NWNNRFLNHC LEHLVQYRTD WDHSWTEQSV DYRHKFSLPS VDGQKRYTFR
VRSRFNPLCG SAQHWSEWSH PIHWGSNTSK ENPFLFALEA
IL-2Rα extracellular domain
SEQ ID NO: 7
ELCDDDPPEI PHATFKAMAY KEGTMLNCEC KRGFRRIKSG SLYMLCTGNS
SHSSWDNQCQ CTSSATRNTT KQVTPQPEEQ KERKTTEMQS PMQPVDQASL
PGHCREPPPW ENEATERIYH FVVGQMVYYQ CVQGYRALHR GPAESVCKMT
HGKTRWTQPQ LICTGEMETS QFPGEEKPQA SPEGRPESET SCLVTTTDFQ
IQTEMAATME TSIFTTEYQ
IgG1FC (with LALA and Knob)-IL-2-T3A/C125S
SEQ ID NO: 8

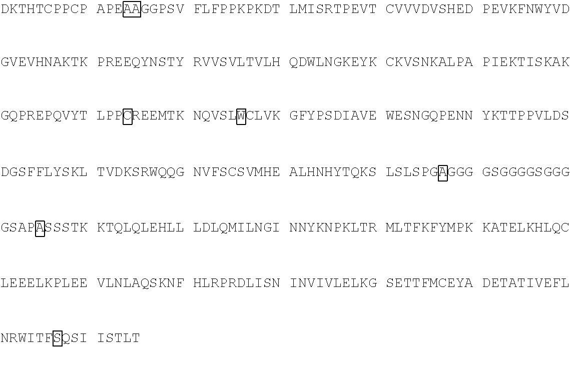

IgG1FC (with LALA and Knob)-IL-2-T3A/C125S/N88R
SEQ ID NO: 9

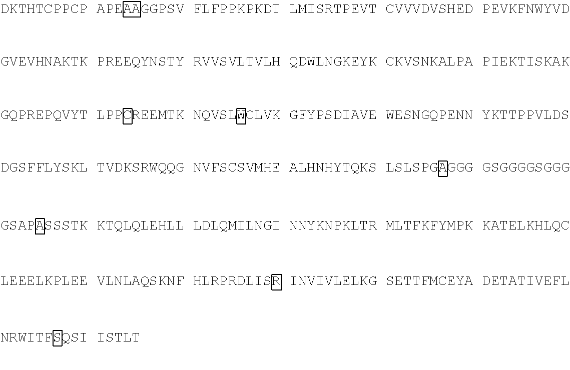

IgG1FC (with LALA and Knob)-IL-2-T3A/C1255/V91K
SEQ ID NO: 10

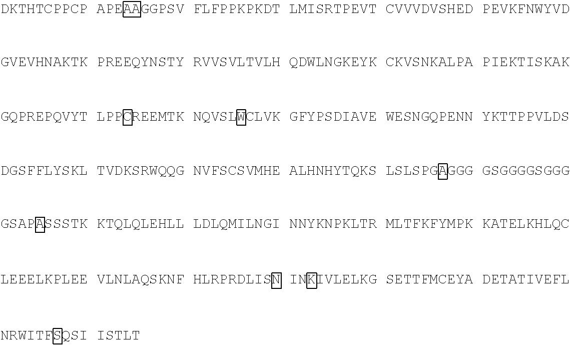

IgG1FC (with LALA and Knob)-IL-2-T3A/C1255/Q126N
SEQ ID NO: 11

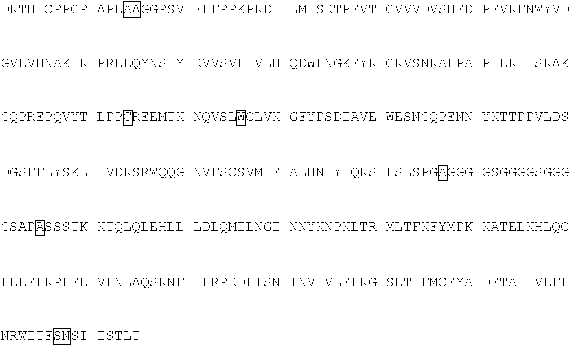

IgG1FC (with LALA/YTE and Knob)-IL-2-T3A/C1255
SEQ ID NO: 12

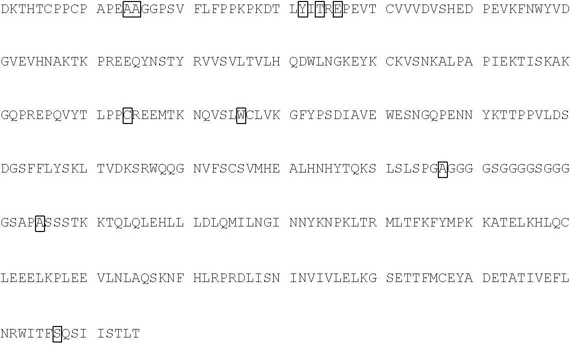

IgG1FC (with LALA/YTE and Knob)-IL-2-T3A/C125S/N88R
SEQ ID NO: 13

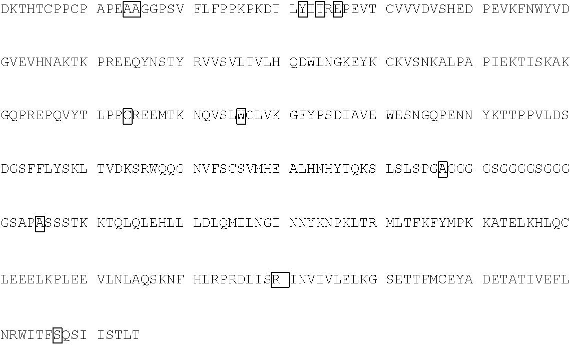

IgG1FC (with LALA/YTE and Knob)-IL-2-T3A/C1255/V91K
SEQ ID NO: 14

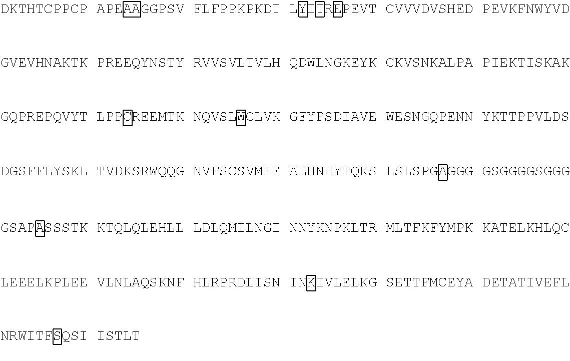

IgG1FC (with LALA/YTE and Knob)-IL-2-T3A/C1255/Q126N
SEQ ID NO: 15

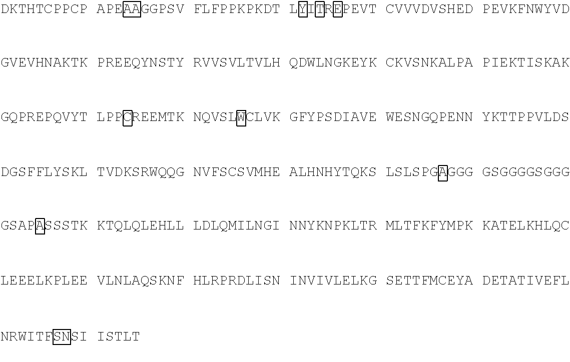

IgG1Fc with LALA/Hole/IL-2Rbeta
SEQ ID NO: 16

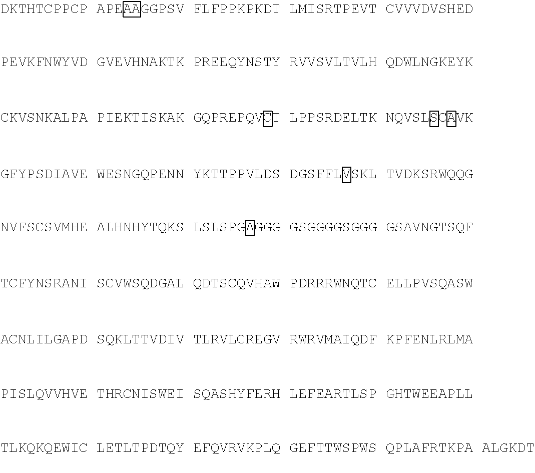

IgG1Fc with LALA/Hole/IL-2Rbeta/ Cleavable
linker
SEQ ID NO: 17

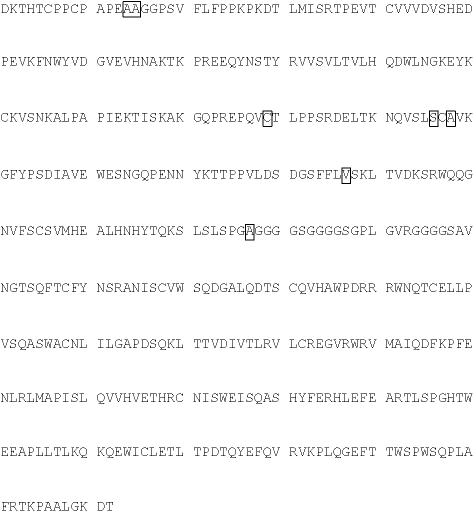

IgG1Fc with LALA/Hole/IL2Rbeta/IL2Rgamma
SEQ ID NO: 18

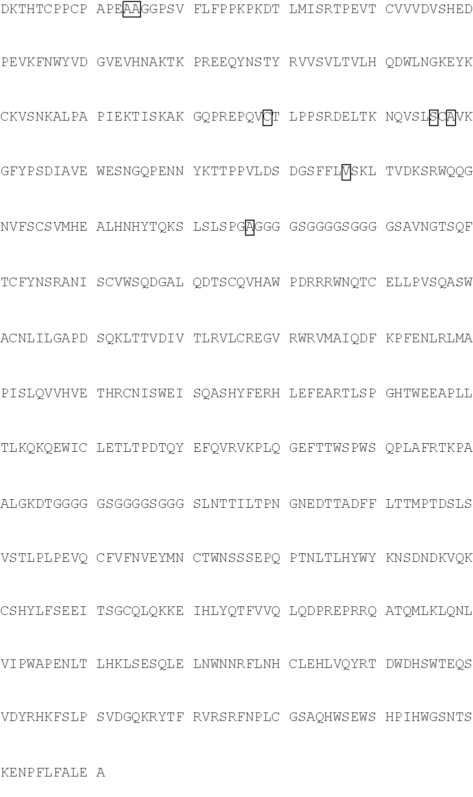

IgG1Fc with LALA/Hole/IL2Rgamma/ IL2Rbeta
SEQ ID NO: 19

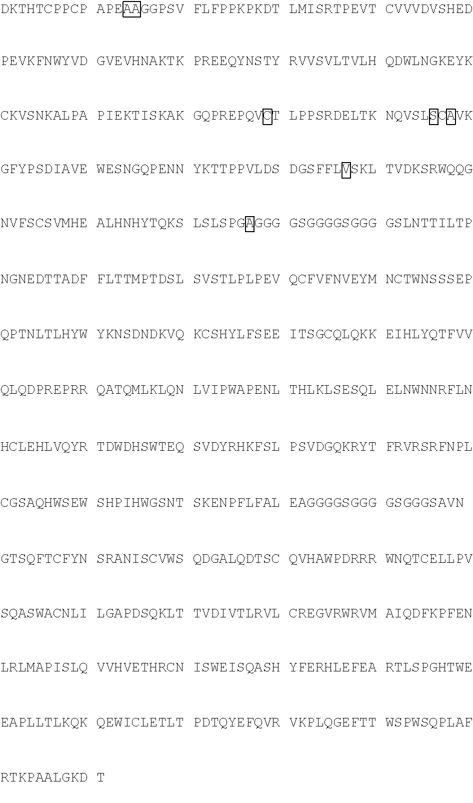

IgG1Fc with LALA/Hole/IL2Rbeta/cleavable
linker/IL2Rgamma
SEQ ID NO: 20

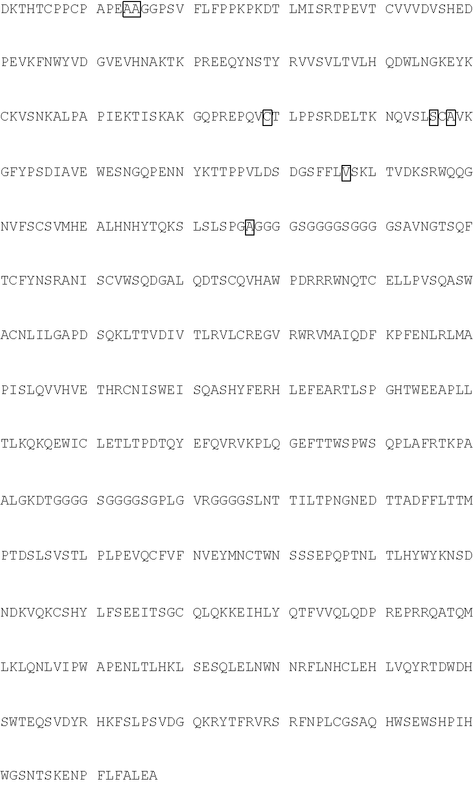

IgG1Fc with LALA/Hole/IL2Rgamma/ cleavable
1inker/IL2Rbeta
SEQ ID NO: 21

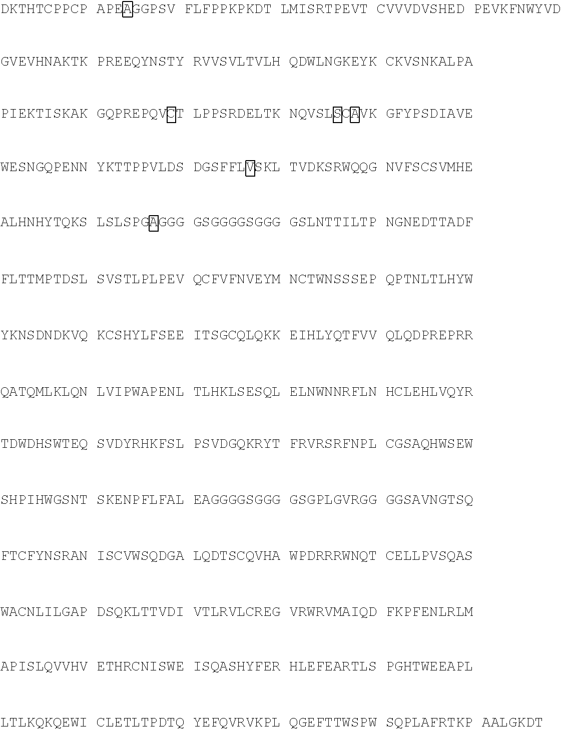

IgG1Fc with YTE/LALA/Hole/IL-2Rbeta
SEQ ID NO: 22

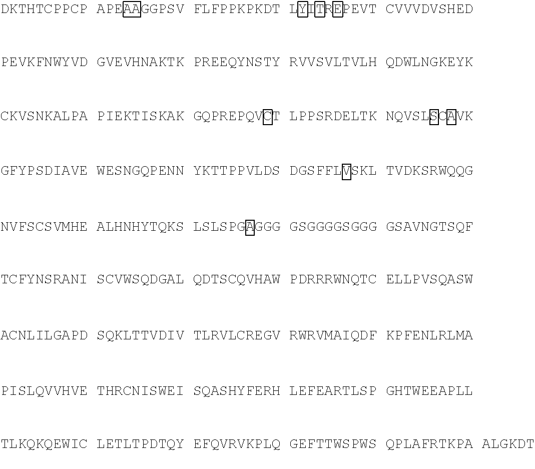

IgG1Fc with YTE/LALA/Hole/IL-2Rbeta/ Cleavable
linker
SEQ ID NO: 23

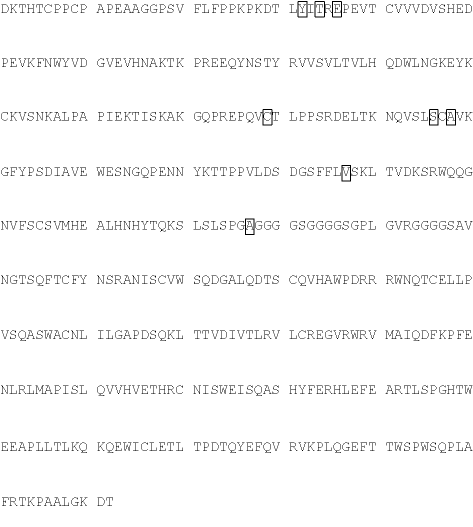

IgG1Fc with YTE/LALA/Hole/IL2Rbeta/IL2Rgamma
SEQ ID NO: 24

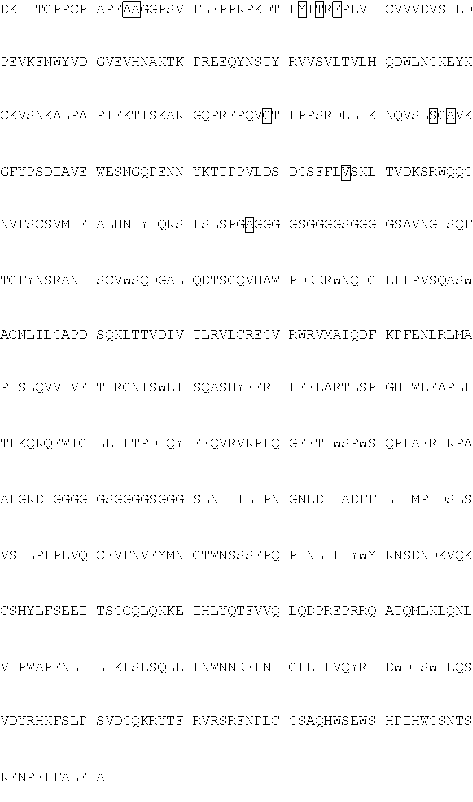

IgG1Fc with YTE/LALA/Hole/IL2Rgamma/ IL2Rbeta
SEQ ID NO: 25

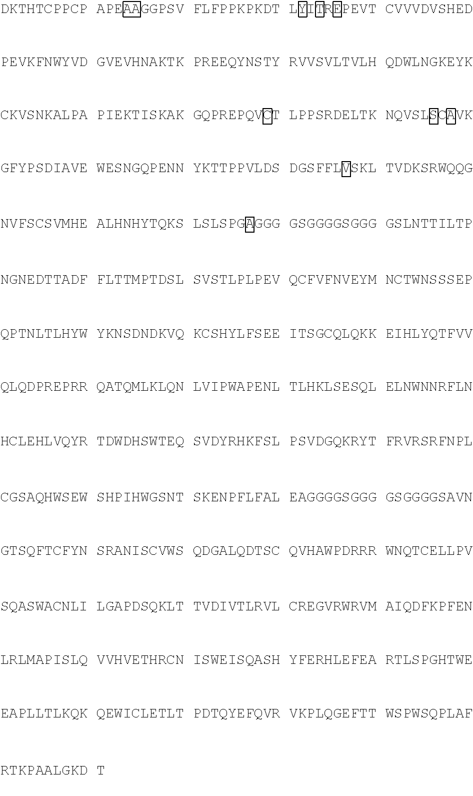

IgG1Fc with YTE/LALA/Hole/IL2Rbeta/cleavable
linker/IL2Rgamma
SEQ ID NO: 26

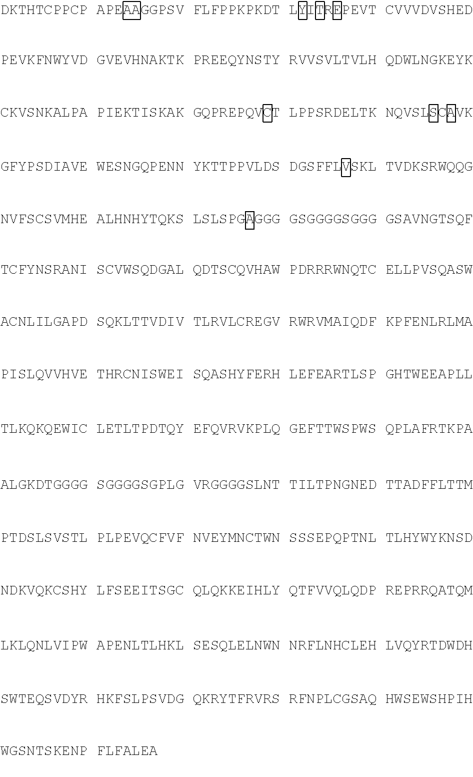

IgG1Fc with YTE/LALA/Hole/IL2Rgamma/ cleavable
1inker/IL2Rbeta
SEQ ID NO: 27

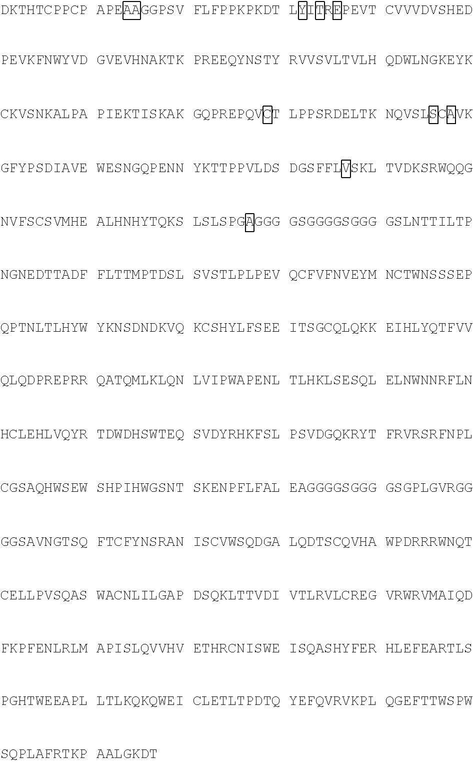

IgG1FC (with LALA and Knob)-IL-2-T3A/C125S/Q126G
SEQ ID NO: 28

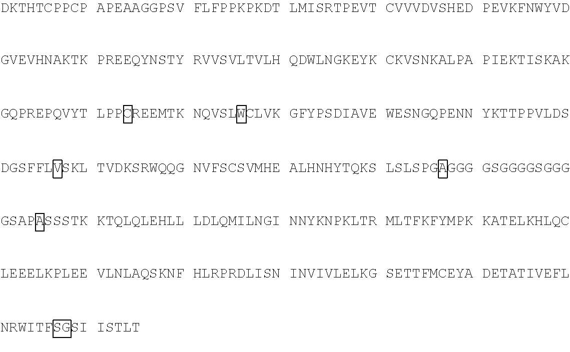

IgG1FC (with LALA and Knob)-IL-2-T3A/C1255/Q126E
SEQ ID NO: 29

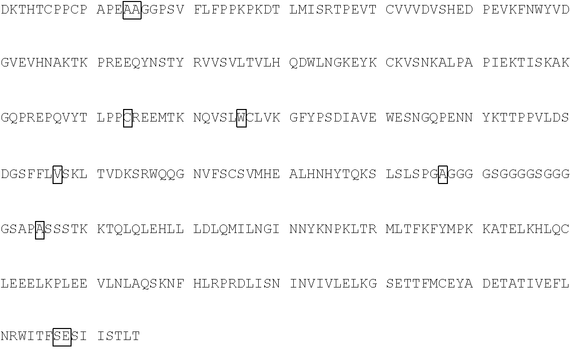

IgG1FC (with LALA and Knob)-IL-2-T3A/C125S/192T
SEQ ID NO: 30

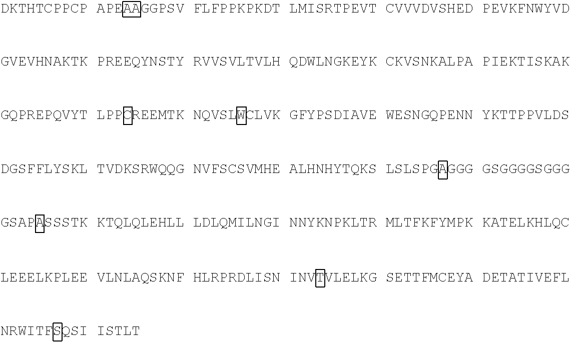

IgG1FC (with LALA/YTE and Knob)-IL-2-T3A/C1255/Q126G
SEQ ID NO: 31

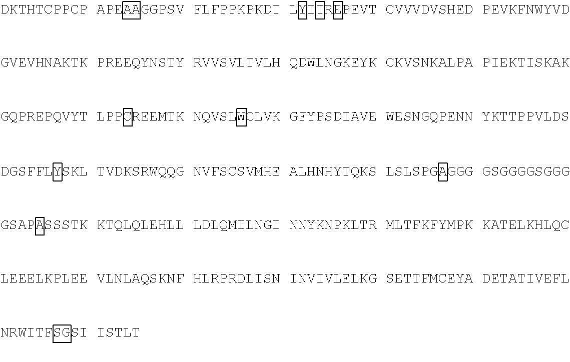

IgG1FC (with LALA/YTE and Knob)-IL-2-T3A/C1255/Q126E
SEQ ID NO: 32

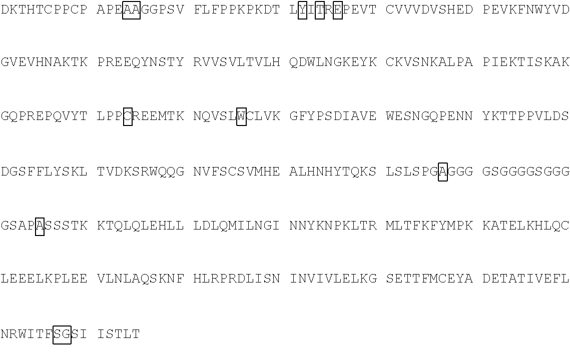

IgG1FC (with LALA/YTE and Knob)-IL-2-T3A/C1255/192T
SEQ ID NO: 33

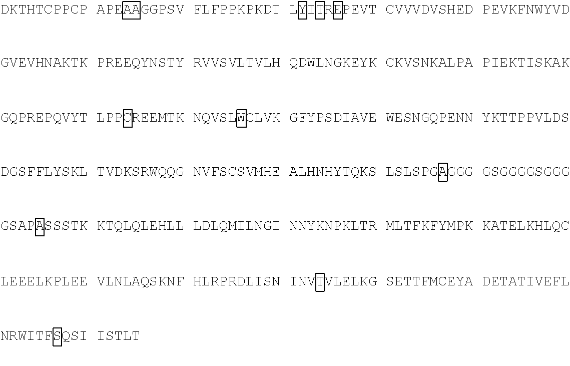

IL-2-T3A/C125S/N88R
SEQ ID NO: 34

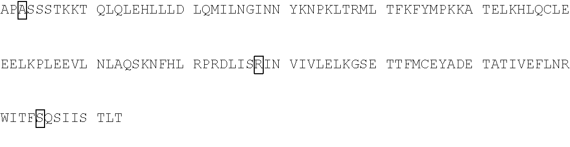

IL-2-T3A/C125S/V91K
SEQ ID NO: 35

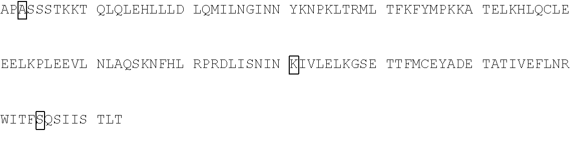

IL-2-T3A/C1255/Q126N
SEQ ID NO: 36

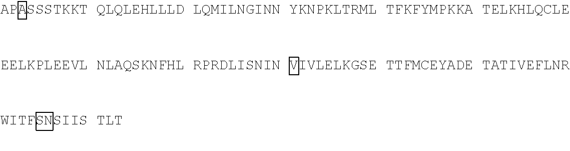

IL-2-T3A/C1255/Q126G
SEQ ID NO: 37

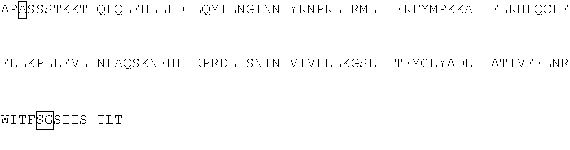

IL-2-T3A/C125S/Q126E
SEQ ID NO: 38

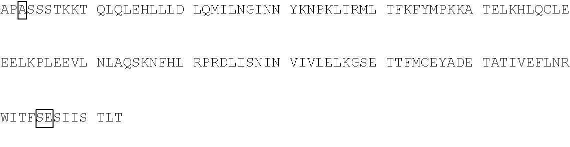

IL-2-T3A/C125S/192T
SEQ ID NO: 39

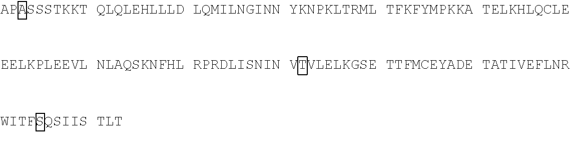

Non-cleavable Peptide Linker
SEQ ID NOs:40-46
(SEQ ID NO: 40)
GGGGS
(SEQ ID NO: 41)
GGGGSGGGGS
(SEQ ID NO: 42)
GGGGSGGGGS GGGGS
(SEQ ID NO: 43)
GGGGSGGGGX GGGGSGGGGS, X = A or N
(SEQ ID NO: 44)
GGGGSGGGGX GGGGYGGGGS, X = S, A or N, and Y = A
or N
(SEQ ID NO: 45)
GGGGSGGGGS AAGGGGSGGG GS
(SEQ ID NO: 46)
GGGGSGGGGS GGGGSAAGGG GSGGGGSGGG GSSRGGGGSG GGGS
cleavable peptide linker
SEQ ID NOs:47-49
(SEQ ID NO: 47)
GPLGVR
(SEQ ID NO: 48)
GPANVR
(SEQ ID NO: 49)
GPASGE
982_CX7_56_5, IgG4 Fc-IL2 (C125A), knob chain
SEQ ID NO: 50

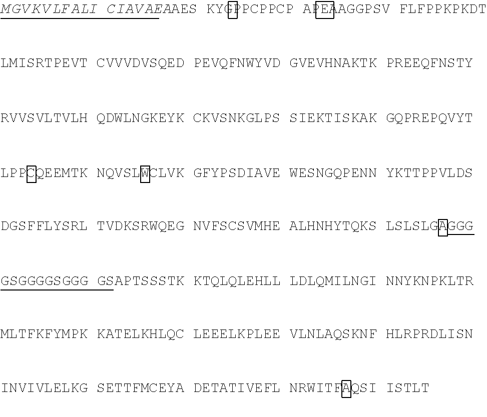

982_CX7_56_5, IgG4 Fc-IL2 (C125A), knob chain
SEQ ID NO: 51

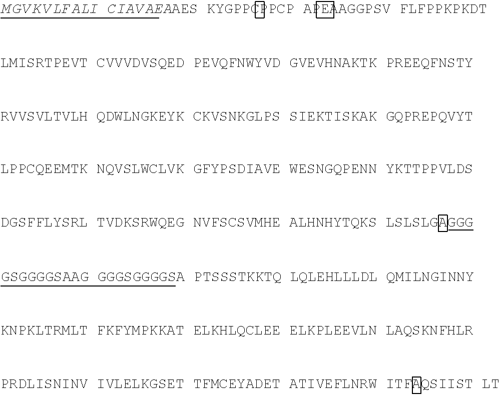

982_CX7_72_2, Fc-IGG4-knob-2xG4SAA2xG4S-
IL2(C125S,V69A/Q74P)
SEQ ID NO: 52

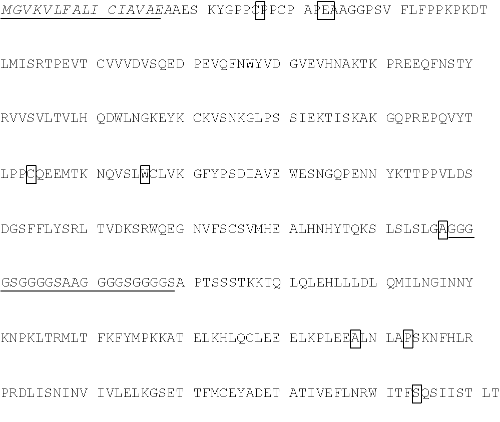

982_CX7_56_6, IgG4 Fc - IL2Rβ,-ECD with long
linker, Hole Chain
SEQ ID NO: 53

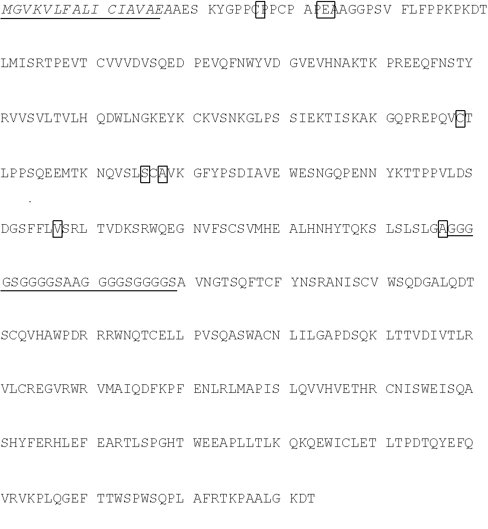

982_CX7_56_4, Hole Chain with a longer peptide linker between
gamma and beta ECDs
SEQ ID NO: 54

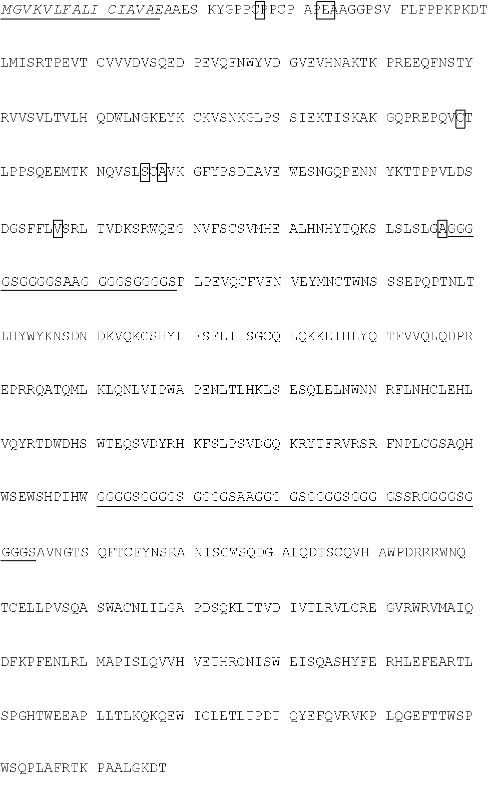

(SEQ ID NO: 55)
GGGSGPASGE GGGGS
(SEQ ID NO: 56)
GGGGSGGGSG PASGEGGGGS
(SEQ ID NO: 57)
GGGGSGGGSG PASGEGGGGS GGGGS
982-Ref with IL-2 mutein comprising mutations V91K and C125A
SEQ ID NO: 58

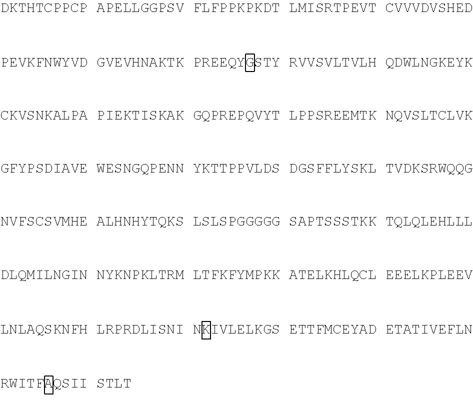

(G4S)₂AA(G4S)₂ linker
SEQ ID NO: 59
GGGGSGGGGS AAGGGGSGGGG S

1. An isolated IL-2 fusion molecule, comprising a carrier moiety, a cytokine moiety, and one or more masking moieties, wherein
the cytokine moiety is fused to the carrier moiety or to a masking moiety,
the one or more masking moieties are fused to the carrier moiety or to the cytokine moiety,
the cytokine moiety comprises an IL-2 polypeptide comprising (i) a C125A or C125S substitution, or (ii) an IL-2 amino acid sequence comprising one or more substitutions selected from T3A, C125S, V69A, and Q74P (numbering according to SEQ ID NO: 1),
the one or more masking moieties bind to the cytokine moiety and inhibit binding of the cytokine moiety to IL-2Rβ and/or IL-2Rγ, but not to IL-2Rα, on immune cells

2. A method of treating an inflammatory condition or an autoimmune disease, comprising administering to a subject in need thereof a therapeutically amount of an isolated IL-2 fusion molecule comprising a carrier moiety, a cytokine moiety and one or more masking moieties, wherein
the cytokine moiety is fused to the carrier moiety or to a masking moiety,
the one or more masking moieties are fused to the carrier moiety or to the cytokine moiety,
the cytokine moiety comprises an IL-2 polypeptide, and
the one or more masking moieties bind to the cytokine moiety and inhibit binding of the cytokine moiety to IL-2Rβ and/or IL-2Rγ, but not to IL-2Rα, on immune cells.

3. The method of claim 2, wherein the inflammatory condition or autoimmune disease is selected from the group consisting of asthma, Type I diabetes, rheumatoid arthritis, allergy, systemic lupus erythematosus, multiple sclerosis, organ graft rejection, and graft-versus-host disease.

4. The IL-2 fusion molecule of claim 1, or the method of claim 2 or 3, wherein the IL-2 fusion molecule has one or more of the following properties:
(a) binds to high affinity IL-2 receptor with alpha, beta, and gamma subunits (IL-2Rαβγ) with an affinity that is at least 100 times higher than that of intermediate IL-2 receptor with beta and gamma subunits (IL-2Rβγ),
(b) binds to IL-2Rβγ with a K_{D} of more than about 5 nM or more than 10 nM as measured in a surface plasmon resonance assay at 37° C.,
(c) has an EC₅₀ value of less than about 1 nM and greater than 0.01 nM, 0.25 nM, or 0.05 nM in a CTLL-2 cell proliferation assay,
(d) has an EC₅₀ value of greater than about 0.05 nM, 0.1 nM, 0.25 nM, or 0.5 nM in a NK92 cell proliferation assay,
(e) has an Emax value at least 5 times or at least 10 times lower in a NK92 cell proliferation assay in the presence of a neutralizing CD25 antibody than in the absence of the neutralizing CD25 antibody,
(f) preferentially stimulates FOXP3⁺ T regulatory cells relative to T effector cells or NK cells,
(g) promotes FOXP3⁺ regulatory T cell growth or survival, and
(h) induces STATS phosphorylation in FOXP3⁺ T cells but has a reduced ability to induce phosphorylation of STATS in FOXP3⁻ T cells.

5. The IL-2 fusion molecule or method of any one of claims 1-4, wherein the IL-2 fusion molecule comprises a masking moiety comprising an extracellular domain (ECD) of IL-2Rβ or IL-2Rγ, or a functional analog thereof, wherein the masking moiety is fused to the carrier moiety with or without a peptide linker.

6. The IL-2 fusion molecule or method of any one of claims 1-4, wherein the IL-2 fusion molecule comprises
a first masking moiety comprising an extracellular domain (ECD) of IL-2Rβ or IL-2Rγ, or a functional analog thereof, wherein the first masking moiety is fused to the carrier moiety with or without a peptide linker, and
a second masking moiety comprising an ECD of IL-2Rγ or IL-2Rβ, or a functional analog thereof, wherein the second masking moiety is fused to the cytokine moiety or to the first masking moiety with or without a peptide linker.

7. The IL-2 fusion molecule or method of claim 5 or 6, wherein the IL-2Rβ ECD or its functional analog has an amino acid sequence at least 95% identical to SEQ ID NO: 3.

8. The IL-2 fusion molecule or method of any one of claims 5-7, wherein the IL-2Rγ ECD or its functional analog has an amino acid sequence at least 95% identical to SEQ ID NO: 6.

9. The IL-2 fusion molecule or method of any one of the preceding claims, wherein the IL-2 polypeptide comprises an amino acid sequence that is at least 95% identical to SEQ ID NO:1, optionally wherein the amino acid sequence is SEQ ID NO: 2.

10. The IL-2 fusion molecule or method of any one of the preceding claims, wherein the carrier moiety is selected from a PEG molecule, an albumin, an albumin fragment, an antibody Fc domain, an antibody, or an antigen-binding fragment thereof.

11. The IL-2 fusion molecule or method of any one of the preceding claims, wherein the cytokine moiety is fused to the carrier moiety or a masking moiety through a non-cleavable peptide linker, and the masking moiety is fused to the carrier moiety or the cytokine moiety through a non-cleavable peptide linker.

12. The IL-2 fusion molecule or method of claim 11, wherein the masking moiety is fused to the carrier moiety or the cytokine moiety through a peptide linker comprising at least 16 amino acids, at least 18 amino acids, at least 20 amino acids, at least 22 amino acids, at least 25 amino acids, at least 30, or up to 44 amino acids.

13. The IL2-fusion molecule or method of any one of claims 1-12, wherein the carrier moiety is an antibody Fc domain, and wherein the fusion molecule is a heterodimer comprising
a first polypeptide chain comprising, from N-terminus to C-terminus, a molecular formula selected from F1-L1-E1, F1-L1-E1-L2-E2, and F1-L1-E2-L2-E1, and
a second polypeptide chain comprising, from N-terminus to C-terminus, a molecular formula F2-L3-C,
wherein
F1 and F2 are the subunits of the Fc domain,
L1, L2 and L3 are peptide linkers,
E1 is an IL-2Rβ ECD or a functional analog thereof, and E2 is an IL-2Rγ ECD or a functional analog thereof, and
C is the cytokine moiety.

14. The IL-2 fusion molecule or method of any one of claims 1-12, wherein the carrier moiety is an antibody Fc domain, and wherein the fusion molecule is a heterodimer comprising
a first polypeptide chain comprising, from N-terminus to C-terminus, a molecular formula selected from E1-L1-F1, E1-L1-E2-L2-F1, and E2-L1-E1-L2-F1, and
a second polypeptide chain comprising, from N-terminus to C-terminus, a molecular formula C-L3-F2,
wherein
F1 and F2 are the subunits of the Fc domain,
L1, L2 and L3 are peptide linkers,
E1 is an IL-2Rβ ECD or a functional analog thereof, and E2 is an IL-2Rγ ECD or a functional analog thereof, and
C is the cytokine moiety.

15. The IL-2 fusion molecule or method of any one of claims 1-12, wherein the carrier moiety is an antibody Fc domain, and wherein the fusion molecule is a heterodimer comprising a first polypeptide chain and a second polypeptide chain comprising, from N-terminus to C-terminus, molecular formulae selected from the following pairs:
F1-L1-E1 and F2-L2-C-L3-E2,
F1-L1-E1 and F2-L2-E2-L3-C,
F1-L1-E2 and F2-L2-C-L3-E1,
F1-L1-E2 and F2-L2-E1-L3-C,
E1-L1-F1 and E2-L2-C-L3-F2,
E1-L1-F1 and C-L2-E2-L3-F2,
E2-L1-F1 and E2-L2-C-L3-F2, and
E2-L1-F1 and C-L2-E1-L3-F2,
wherein
F1 and F2 are the subunits of the Fc domain,
L1, L2 and L3 are peptide linkers,
E1 is an IL-2Rβ ECD or a functional analog thereof, and E2 is an IL-2Rγ ECD or a functional analog thereof, and
C is the cytokine moiety.

16. The IL-2 fusion molecule or method of any one of claims 13-15, wherein the peptide linkers L1, L2, and L3 are not cleavable.

17. The IL-2 fusion molecule or method of any of claim 13-16, wherein L1, L2, and L3 independently have an amino acid sequence selected from SEQ ID NOs: 40-46, 55-57 and 59.

18. The IL-2 fusion molecule or method of any of claims 13-17, wherein at least one of L1, L2, and L3 has an amino acid sequence comprising 20-44 amino acids.

19. The IL-2 fusion molecule or method of any one of claims 13-18, wherein the IL-2 fusion molecule comprises
a first polypeptide chain comprising an amino acid sequence at least 99% identical to SEQ ID NO: 50, 51, or 52, and
a second polypeptide chain comprising an amino acid sequence at least 99% identical to SEQ ID NO: 53 or 54.

20. The IL-2 fusion molecule or method of claim 19, wherein the IL-2 fusion molecule comprises
(a) a first polypeptide chain comprising an amino acid sequence at least 99% identical to SEQ ID NO: 50, and a second polypeptide chain comprising an amino acid sequence at least 99% identical to SEQ ID NO: 53, or
(b) a first polypeptide chain comprising SEQ ID NO: 50, and a second polypeptide chain comprising SEQ ID NO: 53.

21. The IL-2 fusion molecule or method of any one of claims 1-20, wherein the fusion molecule comprises at least two masking moieties, one of which is an ECD of IL-2Rα or a functional analog thereof, wherein the IL-2Rα ECD masking moiety is fused to the cytokine moiety, the carrier moiety, or another masking moiety through a cleavable peptide linker.

22. The IL-2 fusion molecule or method of claim 21, where the IL-2Rα ECD moiety comprises an amino acid sequence at least 95% identical to SEQ ID NO: 7.

23. A polynucleotide encoding the IL-2 fusion molecule of any one of claims 1 and 4-22.

24. An expression vector comprising the polynucleotide of claim 23.

25. A host cell comprising the expression vector of claim 24.

26. A pharmaceutical composition comprising the IL-2 fusion molecule of any one of claims 1 and 4-22 and a pharmaceutically acceptable excipient.

27. The IL-2 fusion molecule of any one of claims 1 and 4-22 or the pharmaceutical composition of claim 26 for use in treating a subject in the method of claim 2 or 3.

28. Use of the IL-2 fusion molecule of any one of claims 1 and 4-22 for the manufacture of a medicament for treating a subject in the method of claim 2 or 3.


SEQUENCE LISTING (59 sequences)

SEQ ID NO: 1 - protein, 133 aa, Homo sapiens [sequence omitted]

SEQ ID NO: 2 - protein, 133 aa, Homo sapiens
  VARIANT (3)..(3): /replace=“Ala”
  VARIANT (125)..(125): /replace=“Ser” or “Gly”
  SITE (1)..(133): /note=“Variant residues given in the sequence have no preference with respect to those in the annotations for variant positions”
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 3 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 4 - protein, 214 aa, Homo sapiens
AVNGTSQFTCFYNSRANISCVWSQDGALQDTSCQVHAWPDRRRWNQTCELLPVSQASWACNLILGAPESQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 5 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFQRRLEFEARTLSPGHTWEEAPLLTLKQKQEWICLETLTPDTQYEFQVRVKPLQGEFTTWSPWSQPLAFRTKPAALGKDT

SEQ ID NO: 6 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 7 - protein, 219 aa, Unknown
  source: /note=“Description of Unknown IL-2Ralpha extracellular domain”
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 8 - protein, 375 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 9 - protein, 375 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLT

SEQ ID NO: 10 - protein, 375 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFSQSIISTLT

SEQ ID NO: 11 - protein, 375 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSNSIISTLT

SEQ ID NO: 12 - protein, 375 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 13 - protein, 375 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLT

SEQ ID NO: 14 - protein, 375 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFSQSIISTLT

SEQ ID NO: 15 - protein, 375 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSNSIISTLT

SEQ ID NO: 16 - protein, 456 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 17 - protein, 462 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGPLGVRGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 18 - protein, 711 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGGSGGGGSGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 19 - protein, 711 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 20 - protein, 717 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGPLGVRGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 21 - protein, 717 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAGGGGSGGGGSGPLGVRGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 22 - protein, 456 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 23 - protein, 462 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGPLGVRGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 24 - protein, 711 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGGSGGGGSGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 25 - protein, 711 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 26 - protein, 717 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGPLGVRGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 27 - protein, 717 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGAGGGGSGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAGGGGSGGGGSGPLGVRGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 28 - protein, 375 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSGSIISTLT

SEQ ID NO: 29 - protein, 375 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSESIISTLT

SEQ ID NO: 30 - protein, 375 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVTVLELKGSETTFMCEYADETATIVEFLNRWITFSQSIISTLT

SEQ ID NO: 31 - protein, 375 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSGSIISTLT

SEQ ID NO: 32 - protein, 375 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSESIISTLT

SEQ ID NO: 33 - protein, 375 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVTVLELKGSETTFMCEYADETATIVEFLNRWITFSQSIISTLT

SEQ ID NO: 34 - protein, 133 aa, Artificial Sequence
  source: /note=“Description of Artificial Sequence Synthetic polypeptide”
APASSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLT

SEQ ID NO: 35 - protein, 133 aa, Artificial Sequence
  source: /note=“Description of Artificial Sequence Synthetic polypeptide”
APASSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFSQSIISTLT

SEQ ID NO: 36 - protein, 133 aa, Artificial Sequence
  source: /note=“Description of Artificial Sequence Synthetic polypeptide”
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSNSIISTLT

SEQ ID NO: 37 - protein, 133 aa, Artificial Sequence
  source: /note=“Description of Artificial Sequence Synthetic polypeptide”
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSGSIISTLT

SEQ ID NO: 38 - protein, 133 aa, Artificial Sequence
  source: /note=“Description of Artificial Sequence Synthetic polypeptide”
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSESIISTLT

SEQ ID NO: 39 - protein, 133 aa, Artificial Sequence
  source: /note=“Description of Artificial Sequence Synthetic polypeptide”
APASSSTKKTQLQLEHLLLDLQMILNGINNYKNPKLTRMLTFKFYMPKKATELKHLQCLEEELKPLEEVLNLAQSKNFHLRPRDLISNINVTVLELKGSETTFMCEYADETATIVEFLNRWITFSQSIISTLT

SEQ ID NO: 40 - protein, 5 aa, Artificial Sequence
  source: /note=“Description of Artificial Sequence Synthetic peptide”
GGGGS

SEQ ID NO: 41 - protein, 10 aa, Artificial Sequence
  source: /note=“Description of Artificial Sequence Synthetic peptide”
GGGGSGGGGS

SEQ ID NO: 42 - protein, 15 aa, Artificial Sequence
  source: /note=“Description of Artificial Sequence Synthetic peptide”
GGGGSGGGGSGGGGS

SEQ ID NO: 43 - protein, 20 aa, Artificial Sequence
  source: /note=“Description of Artificial Sequence Synthetic peptide”
  VARIANT (10)..(10): /replace=“Asn”
  SITE (1)..(20): /note=“Variant residues given in the sequence have no preference with respect to those in the annotations for variant positions”
GGGGSGGGGAGGGGSGGGGS

SEQ ID NO: 44 - protein, 20 aa, Artificial Sequence
  source: /note=“Description of Artificial Sequence Synthetic peptide”
  VARIANT (10)..(10): /replace=“Ala” or “Asn”
  VARIANT (15)..(15): /replace=“Asn”
  SITE (1)..(20): /note=“Variant residues given in the sequence have no preference with respect to those in the annotations for variant positions”
GGGGSGGGGSGGGGAGGGGS

SEQ ID NO: 45 - protein, 22 aa, Artificial Sequence
  source: /note=“Description of Artificial Sequence Synthetic peptide”
GGGGSGGGGSAAGGGGSGGGGS

SEQ ID NO: 46 - protein, 44 aa, Artificial Sequence
  source: /note=“Description of Artificial Sequence Synthetic polypeptide”
GGGGSGGGGSGGGGSAAGGGGSGGGGSGGGGSSRGGGGSGGGGS

SEQ ID NO: 47 - protein, 6 aa, Artificial Sequence
  source: /note=“Description of Artificial Sequence Synthetic peptide”
GPLGVR

SEQ ID NO: 48 - protein, 6 aa, Artificial Sequence
  source: /note=“Description of Artificial Sequence Synthetic peptide”
GPANVR

SEQ ID NO: 49 - protein, 6 aa, Artificial Sequence
  source: /note=“Description of Artificial Sequence Synthetic peptide”
GPASGE

SEQ ID NO: 50 - protein, 395 aa, Artificial Sequence
  source: /note=“Description of Artificial Sequence Synthetic polypeptide”
MGVKVLFALICIAVAEAAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 51 - protein, 402 aa, Artificial Sequence
  source: /note=“Description of Artificial Sequence Synthetic polypeptide”
MGVKVLFALICIAVAEAAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSAAGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 52 - protein, 402 aa, Artificial Sequence
  source: /note=“Description of Artificial Sequence Synthetic polypeptide”
MGVKVLFALICIAVAEAAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSAAGGGGSGGGGSAPTSSSTKKTQLQLEHLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLT

SEQ ID NO: 53 - protein, 483 aa, Artificial Sequence
  source: /note=“Description of Artificial Sequence Synthetic polypeptide”
MGVKVLFALICIAVAEAAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSAAGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 54 - protein, 718 aa, Artificial Sequence
  source: /note=“Description of Artificial Sequence Synthetic polypeptide”
MGVKVLFALICIAVAEAAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSAAGGGGSGGGGSPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGGGGSGGGGSGGGGSAAGGGGSGGGGSGGGGSSRGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 55 - protein, 15 aa, Artificial Sequence
  source: /note=“Description of Artificial Sequence Synthetic peptide”
GGGSGPASGEGGGGS

SEQ ID NO: 56 - protein, 20 aa, Artificial Sequence
  source: /note=“Description of Artificial Sequence Synthetic peptide”
GGGGSGGGSGPASGEGGGGS

SEQ ID NO: 57 - protein, 25 aa, Artificial Sequence
  source: /note=“Description of Artificial Sequence Synthetic peptide”
GGGGSGGGSGPASGEGGGGSGGGGS

SEQ ID NO: 58 - protein, 364 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAPTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFAQSIISTLT

SEQ ID NO: 59 - protein, 21 aa, Artificial Sequence
  source: /note=“Description of Artificial Sequence Synthetic peptide”
GGGGSGGGGSAGGGGSGGGGS